SEQUENCE LISTING (44 sequences)

SEQ ID NO: 1 - protein, 830 aa, Wheat
MTKRLVLFAAVVVALVALTAAEGEASGQLQCERELQEHSLKACRQVVDQQLRDVSPECQPVGGGPVARQYEQQVVVPPKGGSFYPGETTPPQQLQQSILWGIPALLRRYYLSVTSPQQVSYYPGQASSQRPGQGQQPGQGQQEYYLTSPQQSGQWQQPGQGQAGYYPTSPQQSGQEQPGYYPTSPWQPEQLQQPTQGQQRQQPGQGQQLRQGQQGQQSGQGQPRYYPTSSQQPGQLQQLAQGQQGQQPERGQQGQQSGQGQQLGQGQQGQQPGQKQQSGQGQQGYYPISPQQLGQGQQSGQGQLGYYPTSPQQSGQGQSGYYPTSAQQPGQLQQSTQEQQLGQEQQDQQSGQGRQGQQSGQRQQDQQSGQGQQPGQRQPGYYSTSPQQLGQGQPRYYPTSPQQPGQEQQPRQLQQPEQGQQGQQPEQGQQGQQPGQGEQGQQPGQGQQGQQPGQGQPGYYPTSPQQSGQGQPGYYPTSPQQSGQLQQPAQGQQPGQEQQGQQPGQGQQGQQPGQGQQPGQGQPGYYPTSPQQSGQEQQLEQWQQSGQGQPGHYPTSPLQPGQGQPGYYPTSPQQIGQGQQPGQLQQPTQGQQGQQPGQGQQGQQPGQGQQGQQPGQGQQPGQGQPGYYPTSLQQSGQGQQPGQWQQPGQGLPGYYPTSSLQPEQGQQGYYPTSQQQPGQGPQPGQWQQSGQGQQGYYPTSPQQSGQGQQPGQWLQPGQWLQSGYYLTSPQQLGQGQQPRQWLQPRQGQQGYYPTSPQQSGQGQQLGQGQQGYYPTSPQQSGQGQQGYDSPYHVSAEHQAASLKVAKAQQLAAQLPAMCRLEGGDALLASQ

SEQ ID NO: 2 - protein, 815 aa, Wheat
MTKRLVLFAAVVVALVALTAAEGEASGQLQCERELQEHSLKACRQVVDQQLRDVSPECQPVGGGPVARQYEQQVVVPPKGGSFYPGETTPPQQLQQSILWGIPALLRRYYLSVTSPQQVSYYPGQASSQRPGQGQQEYYLTSPQQSGQWQQPGQGQSGYYPTSPQQSGQKQPGYYPTSPWQPEQLQQPTQGQQRQQPGQGQQLRQGQQGQQSGQGQPRYYPTSSQQPGQLQQLAQGQQGQQPERGQQGQQSGQGQQLGQGQQGQQPGQKQQSGQGQQGYYPISPQQLGQGQQSGQGQLGYYPTSPQQSGQGQSGYYPTSAQQPGQLQQSTQEQQLGQEQQDQQSGQGRQGQQSGQRQQDQQSGQGQQPGQRQPGYYSTSPQQLGQGQPRYYPTSPQQPGQEQQPRQLQQPEQGQQGQQPEQGQQGQQQRQGEQGQQPGQGQQGQQPGQGQPGYYPTSPQQSGQGQPGYYPTSPQQSGQLQQPAQGQQPGQEQQGQQPGQGQQPGQGQPGYYPTSPQQSGQEQQLEQWQQSGQGQPGHYPTSPLQPGQGQPGYYPTSPQQIGQGQQPGQLQQPTQGQQGQQPGQGQQGQQPGEGQQGQQPGQGQQPGQGQPGYYPTSLQQSGQGQQPGQWQQPGQGQPGYYPTSSLQPEQGQQGYYPTSQQQPGQGPQPGQWQQSGQGQQGYYPTSPQQSGQGQQPGQWLQPGQWLQSGYYLTSPQQLGQGQQPRQWLQPRQGQQGYYPTSPQQSGQGQQLGQGQQGYYPTSPQQSGQGQQGYDSPYHVSAEHQAASLKVAKAQQLAAQLPAMCRLEGGDALLASQ

SEQ ID NO: 3 - protein, 839 aa, Wheat
MAKRLVLFVAVVVALVALTVAEGEASEQLQCERELQELQERELKACQQVMDQQLRDISPECHPVVVSPVAGQYEQQIVVPPKGGSFYPGETTPPQQLQQRIFWGIPALLKRYYPSVTCPQQVSYYPGQASPQRPGQGQQPGQGQQGYYPTSPQQPGQWQQPEQGQQGYYPTSPQQPGQLQQPAQGQQPGQGQQGQQPGQGQPGYYPTSSQLQPGQLQQPAQGQQGQQPGQAQQGQQPGQGQQPGQGQQGQQPGQGQQPGQGQQGQQLGQGQQGYYPTSLQQSGQGQPGYYPTSLQQLGQGQSGYYPTSPQQPGQGQQPGQLQQPAQGQQPGQGQQGQQPGQGQQGQQPGQGQQPGQGQPGYYPTSPQQSGQGQPGYYPTSSQQPTQSQQPGQGQQGQQVGQGQQAQQPGQGQQPGQGQPGYYPTSPQQSGQGQPGYYLTSPQQSGQGQQPGQLQQSAQGQKGQQPGQGQQPGQGQQGQQPGQGQQGQQPGQGQPGYYPTSPQQSGQGQQPGQWQQPGQGQPGYYPTSPLQPGQGQPGYDPTSPQQPGQGQQPGQLQQPAQGQQGQQLAQGQQGQQPAQVQQGQQPAQGQQGQQLGQGQQGQQPGQGQQGQQPAQGQQGQQPGQGQHGQQPGQGQQGQQPGQGQQPGQGQPWYYPTSPQESGQGQQPGQWQQPGQGQPGYYLTFSVAARTGQQGYYPTSLQQPGQGQQPGQWQQSGQGQHWYYPTSPKLSGQGQRPGQWLQPGQGQQGYYPTSPQQPPQGQQLGQWLQPGQGQQGYYPTSLQQTGQGQQSGQGQQGYYSSYHVSVEHQAASLKVAKAQQLAAQLPAMCRLEGGDALSASQ

SEQ ID NO: 4 - protein, 838 aa, Wheat
MAKRLVLFVAVVVALVALTVAEGEASEQLQCERELQELQERELKACQQVMDQQLRDISPECHPVVVSPVAGQYEQQIVVPKGGSFYPGETTPPQQLQQRIFWGIPALLKRYYPSVTSPQQVSYYPGQASPQRPGQGQQPGQGQQSGQGQQGYYPTSPQQPGQWQQPEQGQPGYYPTSPQQPGQLQQPAQGQQPGQGQQGRQPGQGQPGYYPTSSQLQPGQLQQPAQGQQGQQPGQGQQGQQPGQGQQPGQGQQGQQPGQGQQPGQGQQGQQLGQGQQGYYPTSLQQSGQGQPGYYPTSLQQLGQGQSGYYPTSPQQPGQGQQPGQLQQPAQGQQPEQGQQGQQPGQGQQGQQPGQGQQPGQGQPGYYPTSPQQSGQGQPGYYPTSSQQPTQSQQPGQGQQGQQVGQGQQAQQPGQGQQPGQGQPGYYPTSPLQSGQGQPGYYLTSPQQSGQGQQPGQLQQSAQGQKGQQPGQGQQPGQGQQGQQPGQGQQGQQPGQGQPGYYPTSPQQSGQGQQPGQWQQPGQGQPGYYPTSPLQPGQGQPGYDPTSPQQPGQGQQPGQLQQPAQGQQGQQLAQGQQGQQPAQVQQGQQPAQGQQGQQLGQGQQGQQPGQGQQPAQGQQGQQPGQGQQGQQPGQGQQPGQGQPWYYPTSPQESGQGQQPGQWQQPGQWQQPGQGQPGYYLTSPLQLGQGQQGYYPTSLQQPGQGQQPGQWQQSGQGQHGYYPTSPQLSGQGQRPGQWLQPGQGQQGYYPTSPQQSGQGQQLGQWLQPGQGQQGYYPTSLQQTGQGQQSGQGQQGYYSSYHVSVEHQAASLKVAKAQQLAAQLPAMCRLEGGDALSASQ

SEQ ID NO: 5 - protein, 789 aa, Wheat
MAKRLVLFAAVVVALVALTAAEGEASGQLQCEHELEACQQVVDQQLRDVSPGCRPITVSPGTRQYEQQPVVPSKAGSFYPSETTPSQQLQQMIFWGIPALLRRYYPSVTSSQQGSYYPGQASPQQSGQGQQPGQEQQPGQGQQDQQPGQRQQGYYPTSPQQPGQGQQLGQGQPGYYPTSQQPGQKQQAGQGQQSGQGQQGYYPTSPQQSGQGQQPGQGQPGYYPTSPQQSGQWQQPGQGQQPGQGQQSGQGQQGQQPGQGQRPGQGQQGYYPISPQQPGQGQQSGQGQPGYYPTSLRQPGQWQQPGQGQQPGQGQQGQQPGQGQQSGQGQQGYYPTSLQQPGQGQQLGQGQPGYYPTSQQSEQGQQPGQGKQPGQGQQGYYPTSPQQSGQGQQLGQGQPGYYPTSPQQSGQGQQSGQGQQGYYPTSPQQSGQGQQPGQGQSGYFPTSRQQSGQGQQPGQGQQSGQGQQGQQPGQGQQAYYPTSSQQSRQRQQAGQWQRPGQGQPGYYPTSPQQPGQEQQSGQAQQSGQWQLVYYPTSPQQPGQLQQPAQGQQPAQGQQSAQEQQPGQAQQSGQWQLVYYPTSPQQPGQLQQPAQGQQGYYPTSPQQSGQGQQGYYPTSPQQSGQGQQGYYPTSPQQSGQGQQPGQGQQPRQGQQGYYPISPQQSGQGQQPGQGQQGYYPTSPQQSGQGQQPGHEQQPGQWLQPGQGQQGYYPTSSQQSGQGHQSGQGQQGYYPTSLWQPGQGQQGYASPYHVSAEYQAARLKVAKAQQLAAQLPAMCRLEGSDALSTRQ

SEQ ID NO: 6 - protein, 660 aa, Wheat
MAKRLVLFAAVVIALVALTTAEGEASRQLQCERELQESSLEACRQVVDQQLAGRLPWSTGLQMRCCQQLRDVSAKCRSVAVSQVARQYEQTVVPPKGGSFYPGETTPLQQLQQGIFWGTSSQTVQGYYPSVTSPRQGSYYPGQASPQQPGQGQQPGKWQEPGQGQQWYYPTSLQQPGQGQQIGKGKQGYYPTSLQQPGQGQQIGQGQQGYYPTSPQHTGQRQQPVQGQQIGQGQQPEQGQQPGQWQQGYYPTSPQQLGQGQQPGQWQQSGQGQQGHYPTSLQQPGQGQQGHYLASQQQPAQGQQGHYPASQQQPGQGQQGHYPASQQQPGQGQQGHYPASQQEPGQGQQGQIPASQQQPGQGQQGHYPASLQQPGQQGHYPTSLQQLGQGQQIGQPGQKQQPGQGQQTGQGQQPEQEQQPGQGQQGYYPTSLQQPGQGQQQGQGQQGYYPTSLQQPGQGQQGHYPASLQQPGQGQGQPGQRQQPGQGQHPEQGQQPGQGQQGYYPTSPQQPGQGQQLGQGQQGYYPTSPQQPGQGQQPGQGQQGHCPMSPQQTGQAQQLGQGQQIGQVQQPGQGQQGYYPTSLQQPGQGQQSGQGQQSGQGHQPGQGQQSGQEKQGYDSPYHVSAEQQAASPMVAKAQQPATQLPTVCRMEGGDALSASQ

SEQ ID NO: 7 - protein, 648 aa, Wheat
MAKRLVLFAAVVIALVALTTAEGEASRQLQCERELQESSLEACRQVVDQQLAGRLPWSTGLQMRCCQQLRDVSAKCRSVAVSQVARQYEQTVVPPKGGSFYPGETTPLQQLQQGIFWGTSSQTVQGYYPGVTSPRQGSYYPGQASPQQPGQGQQPGKWQEPGQGQQWYYPTSLQQPGQGQQIGKGQQGYYPTSLQQPGQGQQGYYPTSLQHTGQRQQPVQGQQPEQGQQPGQWQQGYYPTSPQQLGQGQQPRQWQQSGQGQQGHYPTSLQQPGQGQQGHYLASQQQPGQGQQGHYPASQQQPGQGQQGHYPASQQQPGQGQQGHYPASQQEPGQGQQGQIPASQQQPGQGQQGHYPASLQQPGQGQQGHYPTSLQQLGQGQQTGQPGQKQQPGQGQQTGQGQQPEQEQQPGQGQQGYYPTSLQQPGQGQQQGQGQQGYYPTSLQQPGQGQQGHYPASLQQPGQGQPGQRQQPGQGQHPEQGKQPGQGQQGYYPTSPQQPGQGQQLGQGQQGYYPTSPQQPGQGQQPGQGQQGHCPTSPQQSGQAQQPGQGQQIGQVQQPGQGQQGYYPTSVQQPGQGQQSGQGQQSGQGHQPGQGQQSGQEQQGYDSPYHVSAEQQAASPMVAKAQQPATQLPTVCRMEGGDALSASQ

SEQ ID NO: 8 - protein, 705 aa, Wheat
MAKRLVLFATVVITLVALTAAEGEASRQLQCERELQESSLEACRQVVDQQLAGRLPWSTGLQMRCCQQLRDVSAKCRPVAVSQVVRQYEQTVVPPKGGSFYPGETTPLQQLQQVIFWGTSSQTVQGYYPSVSSPQQGPYYPGQASPQQPGQGQQPGKWQELGQGQQGYYPTSLHQSGQGQQGYYPSSLQQPGQGQQIGQGQQGYYPTSLQQPGQGQQIGQGQQGYYPTSPQHPGQRQQPGQGQQIGQGQQLGQGRQIGQGQQSGQGQQGYYPTSPQQLGQGQQPGQWQQSGQGQQGYYPTSQQQPGQGQQGQYPASQQQPGQGQQGQYPASQQQPGQGQQGQYPASQQQPGQGQQGHYLASQQQPGQGQQRHYPASLQQPGQGQQGHYTASLQQPGQGQQGHYPASLQQVGQGQQIGQLGQRQQPGQGQQTRQGQQLEQGQQPGQGQQTRQGQQLEQGQQPGQGQQGYYPTSPQQSGQGQQPGQSQQPGQGQQGYYSSSLQQPGQGLQGHYPASLQQPGQGHPGQRQQPGQGQQPEQGQQPGQGQQGYYPTSPQQPGQGKQLGQGQQGYYPTSPQQPGQGQQPGQGQQGHCPTSPQQTGQAQQPGQGQQIGQVQQPGQGQQGYYPISLQQSGQGQQSGQGQQSGQGHQLGQGQQSGQEQQGYDNPYHVNTEQQTASPKVAKVQQPATQLPIMCRMEGGDALSASQ

SEQ ID NO: 9 - protein, 602 aa, Wheat
MAKRLVLFATVVIGLVSLTVAEGEASKQLQCERELQESSLEACRLVVDQQLASRLPWSTGLQMRCCQQLRDISAKCRPVALSQVARQYGQTAVPPKGGPFYHRETTPLQQLQQGIFGGTSSQTVQGYYPSVISPQQGSYYPGQASPQQPGKWQELGQGQQWYYPTSLQQPGQGQQGYYRTSLQQPGQRQQGYYRTSLQQPGQGQQIGQWQQGYYPTSPQHPGQGQQPGQVQKIGQGQQPEKGQQLGQEQQIGQGQQPEQGQQPGQGQQPGQGQQGYYPTSLQQPGQGQQPGQWQQPGQGQQGYYPTSLQQPVQGQQGHYPASQHQPGQGQQGHQPASLQLSGQGQQGHHPASLQQPGQGKQTGQREQRQQPGQGQQTGQGQQPEQEQQPGQGQQGYYPTYLQQPGQGQQPEQWQQPGQGQQGHYPASLQQSGQGQQGHYPASLQQLGQGQPGQTQQPGQGQQPEQEEQSGQGQQGYYPTSPQQPGQGQQGHFPTSGQAQQPGQGQQIGQAQQLGQGQQGYYPTSLQQPGQEQQSGQGQQLGQGHQPGQGQQSGQEQQGYDSPYHVSVEQQAASPKVAKAHHPVAQLPTMCQMEGGDALSASQ

SEQ ID NO: 10 - protein, 621 aa, Artificial Sequence
  Consensus sequence derived from wheat sequences of Table 1
MAKRLVLFAAVVVALVALTAEGEASQLQCERELQESLACRQVVDQQLRDVSPCRPVVSPVARQYEQQVVPPKGGSFYPGETTPQQLQQIFWGIPALLRYYPSVTSPQQGSYYPGQASPQQPGQGQQPGQGQQGYYTSPQQPGQQQGQGQGYYPTSQQPGQQQQGQQGQGQPGYYPTSQPGQQQPQGQQQQQGQQGQGQQGQGQQGQQPGQQQGQGQQGQQPQQSGQGQGYYPTSQQPGQGQQQQQQQGQQPGQGQQGQQPGQGQQPGQGQQGYYPTSQQPGQGQQGYPSQQPGQQPQQGQQQPQGQQPGQGQPGYYPTSPQQSGQGQGYYTSPQQSGQQQPQQQGQQGQQPGQGQQPGQGQQGQPGQGQGYYPTSPQQSGQQQPGQWQQPGQGQPGYYPTSPQQPGQGQQGYYPTSPQQPGQGQQPQQQPQGQQQQQQQQPQGQQPGQGQQPGQGQQGYYPTSPQQSGQGQQGQGYYTGQQGYYPTSQQPGQGQQPGQQQQGQYYPSPSGQGQPGQQGYYPTSPQQGQGQQPGQQGQWLQPGQGQQGYYPTSLQQGQGQQSGQGQQGYYPQQSGQQQGYDSPYHVSAEQAASLKVAKAQQLAAQLPAMCRLEGGDALSASQ

SEQ ID NO: 11 - protein, 18 aa, Wheat
LKVAKAQQLAAQLPAMCR

SEQ ID NO: 12 - DNA, 2073 bp, Guinea pig
  CDS (1)..(2073): transglutaminase enzyme
atggcagaggatctgatcctggagagatgtgatttgcagctggaggtcaatggccgcgaccaccgcacggccgacctgtgccgggagaggctggtgttgcggcggggccagcccttctggctgacgctgcactttgagggccgtggctacgaggctggtgtggacactctcaccttcaacgctgtgaccggcccagatcccagtgaggaggccgggactatggcccggttctcactgtccagtgctgtcgaggggggcacctggtcagcctcagcagtggaccagcaggacagcactgtctcgctgctgctcagcaccccagctgatgcccccattggcctgtatcgcctcagcctggaggcctccactggttaccagggctccagcttcgtactgggccacttcatcctgctctacaatcctcggtgcccagcggatgctgtctatatggactcagaccaagagcggcaggagtatgtgctcacccaacagggcttcatctaccagggctcggccaagttcatcaatggcataccttggaacttcgggcagtttgaagatgggatcctggatatttgcctgatgctcttggacaccaaccccaagttcctgaagaatgctggccaagactgctcgcgccgcagcagacctgtctacgtgggccgggtggtgagcgccatggtcaactgcaatgacgatcagggcgtgcttcagggacgctgggacaacaactacagtgatggtgtcagccccatgtcctggatcggcagcgtggacatcctgcggcgctggaaagactatgggtgccagcgcgtcaagtacggccagtgctgggtcttcgctgctgtggcctgcacagtgctgcggtgccttggcatccccacccgagtcgtgaccaactttaactcagcccacgaccagaacagcaacctgctcatcgagtacttccgaaacgagtctggggagatcgaggggaacaagagcgagatgatctggaacttccactgctgggtggagtcgtggatgaccaggccggacctggagcctgggtacgaggggtggcaggccctggaccccacaccccaggagaagagtgaagggacatactgctgtggcccagttccggttcgagccatcaaggagggccacctgaacgtcaagtatgatgcacctttcgtgtttgctgaggtcaatgctgacgtggtgaactggatccggcagaaagatgggtccctgcgcaagtccatcaaccatttggttgtggggctgaagatcagtactaagagtgtgggccgcgatgagcgagaggacatcacccacacctacaagtacccagagggatctgaagaggagcgggaagcttttgttagggccaaccacctaaataaactggccacaaaggaagaggctcaggaggaaacgggagtggccatgcggatccgtgtgggccagaacatgactatgggcagtgactttgacatctttgcctacatcaccaatggcactgctgagagccacgaatgccaactcctgctctgtgcacgcatcgtcagctacaatggagtcctggggcccgtgtgcagcaccaacgacctgctcaacctgaccctggatcccttctcggagaacagcatccccctgcacatcctctatgagaagtacggtgactacctgactgagtccaacctcatcaaggtgcgaggcctccttatcgagccagcagccaacagctatgtattggccgagagggacatttacctggagaatccagaaatcaagatccgggtcttgggggagcccaagcagaaccgcaagctgattgctgaggtgtctctgaagaatccgctccctgtgccgctgctgggttgtatcttcaccgtggaaggagctggcctgaccaaggaccagaagtcggtggaggtcccagaccccgtggaagcaggggagcaagcgaaggtacgggtggacctgctgccgacggaggtgggcctccacaagctggtggtgaacttcgagtgcgacaagctgaaggccgtgaagggctatcggaacgtcatcatcggccccgcctaa

SEQ ID NO: 13 - DNA, 736 bp, Rice
gaattccttctacatcggcttaggtgtagcaacacgactttattattattattattattattattattattttacaaaaatataaaatagatcagtccctcaccacaagtagagcaagttggtgagttattgtaaagttctacaaagctaatttaaaagttattgcattaacttatttcatattacaaacaagagtgtcaatggaacaatgaaaaccatatgacatactataattttgtttttattattgaaattatataattcaaagagaataaatccacatagccgtaaagttctacatgtggtgcattaccaaaatatatatagcttacaaaacatgacaagcttagtttgaaaaattgcaatccttatcacattgacacataaagtgagtgatgagtcataatattattttctttgctacccatcatgtatatatgatagccacaaagttactttgatgatgatatcaaagaacatttttaggtgcacctaacagaatatccaaataatatgactcacttagatcataatagagcatcaagtaaaactaacactctaaagcaaccgatgggaaagcatctataaatagacaagcacaatgaaaatcctcatcatccttcaccacaattcaaatattatagttgaagcatagtagtagaatccaacaacaatgaagatcattttcgtatttgctctccttgctattgttgcatgcaatgcctctgcgtctaga

SEQ ID NO: 14 - DNA, 32 bp, Artificial Sequence
  PLT217 forward primer for amplification of wheat gene Ax1
gctcagcagagttctatcactggctggccaac

SEQ ID NO: 15 - DNA, 31 bp, Artificial Sequence
  PLT219 reverse primer for amplification of wheat gene Ax1
ggatccgattacgtggctttagcagaccgtc

SEQ ID NO: 16 - DNA, 29 bp, Artificial Sequence
  PLT228 forward primer for amplification of wheat gene Ax2
ggatccgcttagaagcattgagtggccgc

SEQ ID NO: 17 - DNA, 31 bp, Artificial Sequence
  PLT230 reverse primer for amplification of wheat gene Ax2
gctcagcctatcactggctggccaacaatgc

SEQ ID NO: 18 - DNA, 29 bp, Artificial Sequence
  PLT185 forward primer for amplification of wheat gene Bx7
tctagaatggcactactcgacatggttag

SEQ ID NO: 19 - DNA, 21 bp, Artificial Sequence
  PLT186 reverse primer for amplification of wheat gene Bx7
caccatgcaagctgcagagag

SEQ ID NO: 20 - DNA, 28 bp, Artificial Sequence
  PLT562 forward primer for amplification of wheat gene Bx17
tctagatatggctaagcggttagtcctc

SEQ ID NO: 21 - DNA, 25 bp, Artificial Sequence
  PLT563 reverse primer for amplification of wheat gene Bx17
gatatctgcgagctgcagagagttc

SEQ ID NO: 22 - DNA, 28 bp, Artificial Sequence
  PLT272 forward primer for amplification of wheat gene By9
cccgggcacagataaatgttgtgattca

SEQ ID NO: 23 - DNA, 27 bp, Artificial Sequence
  PLT273 reverse primer for amplification of wheat gene By9
gtcgactgcaagttgcagagagttcat

SEQ ID NO: 24 - DNA, 27 bp, Artificial Sequence
  G1B5 forward primer for amplification of wheat gene Dx5
tgttccatgcaggctacctcccactac

SEQ ID NO: 25 - DNA, 26 bp, Artificial Sequence
  PLT189 reverse primer for amplification of wheat gene Dx5
gtcgacatgcctaagcaccatgcgag

SEQ ID NO: 26 - DNA, 30 bp, Artificial Sequence
  G2B3 forward primer for amplification of wheat gene Dy10
aagcttttcattttgcattattattgggtt

SEQ ID NO: 27 - DNA, 27 bp, Artificial Sequence
  G2B5 reverse primer for amplification of wheat gene Dy10
accttatccatgcaagctaccttccac

SEQ ID NO: 28 - DNA, 32 bp, Artificial Sequence
  PLT482 forward primer for amplification of wheat gene Dy12
gaattcgcagatttgcaaaagcaatggctaac

SEQ ID NO: 29 - DNA, 34 bp, Artificial Sequence
  PLT483 reverse primer for amplification of wheat gene Dy12
tctagagcttgtgagaaaggggtaatcatcagtg

SEQ ID NO: 30 - DNA, 31 bp, Artificial Sequence
  PLT488 forward primer for amplification of wheat gene HMW2
gaattcagctttgagtggccgtagatttgca

SEQ ID NO: 31 - DNA, 33 bp, Artificial Sequence
  PLT489 reverse primer for amplification of wheat gene HMW2
ggatccatataggatctgtcgcattcatggctg

SEQ ID NO: 32 - DNA, 26 bp, Artificial Sequence
  PLT571 forward primer for amplification of wheat gene Glu1a
tctagatggctaagcggttggtcctc

SEQ ID NO: 33 - DNA, 33 bp, Artificial Sequence
  PLT572 reverse primer for amplification of wheat gene Glu1a
gatatcgctccttgttgcattcaacactcttac

SEQ ID NO: 34 - DNA, 30 bp, Artificial Sequence
  PLT237 forward primer for amplification of guinea pig gene transglutaminase
tctagaatggcagaggatctgatcctggag

SEQ ID NO: 35 - DNA, 28 bp, Artificial Sequence
  PLT238 reverse primer for amplification of guinea pig gene transglutaminase
gagctcttaggcggggccgatgatgacg

SEQ ID NO: 36 - protein, 9 aa, Artificial Sequence
  Sequence derived from wheat storage proteins wherein the allergenic amino acid is eliminated (Gln at position 6 may be mutated)
PFPQPQLPY

SEQ ID NO: 37 - protein, 9 aa, Artificial Sequence
  Sequence derived from wheat storage proteins wherein the allergenic amino acid is eliminated (Gln at position 4 may be mutated)
PQPQLPYPQ

SEQ ID NO: 38 - protein, 9 aa, Artificial Sequence
  Sequence derived from wheat storage proteins wherein the allergenic amino acid is eliminated (Gln at position 6 may be mutated)
PYPQPQLPY

SEQ ID NO: 39 - protein, 13 aa, Artificial Sequence
  Sequence derived from wheat storage proteins wherein the allergenic amino acid is eliminated (Gln at position 10 may be mutated)
LQLQPFPQPQLPY

SEQ ID NO: 40 - protein, 13 aa, Artificial Sequence
  Sequence derived from wheat storage proteins wherein the allergenic amino acid is eliminated (Tyr and Ser at positions 5 and 8 may be mutated)
QQGYYPTSPQQSG

SEQ ID NO: 41 - protein, 8 aa, Artificial Sequence
  Sequence derived from wheat storage proteins wherein the allergenic amino acid is eliminated (Tyr and Ser at positions 5 and 8 may be mutated)
QQGYYPTS

SEQ ID NO: 42 - protein, 8 aa, Artificial Sequence
  Sequence derived from wheat storage proteins wherein the allergenic amino acid is eliminated (Gln at positions 4, 5 and 7 may be mutated)
PFSQQQQQ

SEQ ID NO: 43 - protein, 12 aa, Artificial Sequence
  Sequence derived from wheat storage proteins wherein the allergenic amino acid is eliminated (Gln at positions 4 and 6 may be mutated)
QSEQSQQPFQPQ

SEQ ID NO: 44 - protein, 9 aa, Artificial Sequence
  Sequence derived from wheat storage proteins wherein the allergenic amino acid is eliminated (Gln at position 4 may be mutated)
  misc_feature (2)..(2): Xaa can be any naturally occurring amino acid
QXPQQPQQF

(19) United States
(12) Patent Application Publication
(10) Pub. No.: US 2007/0079403 A1
(43) Pub. Date: Apr. 5, 2007

(54) Food flours with specific technological characteristics and low allegenicity

(76) Inventor: Corrado Fogher, Vicomoscano (IT)

Correspondence Address: NIXON & VANDERHYE, PC, 901 NORTH GLEBE ROAD, 11TH FLOOR, ARLINGTON, VA, 22203, US

(21) Appl. No.: 10/534,742

(22) PCT Filed: Nov. 12, 2003
(86) PCT No.: PCT/IB03/05092

§ 371 Date: Jun. 1, 2005

(51) Int. Cl. A01H 5/00; C12N 5/04; C12N 15/82

(52) U.S. Cl. 800/320.3; 435/419; 435/468

(57) ABSTRACT

A new cereal from which seed a food flour can be obtained having leavening ability and low allergenicity, and from which dough bakery products can be obtained.

### FIELD OF THE ART

The present invention refers to the technology for the realization of a new cereal from which seed a food flour can be obtained, and from which dough bakery products can be obtained.

International Classification: C12 n, A01 b.

### STATE OF THE ART

The bakery products currently known are obtained almost exclusively from wheat flours. Such flours, due to the leavening property of their dough made of water and yeast, maintain an alveolar structure after baking in the oven.

Such a property gives wheat flours, especially when kneaded with water and yeast, the quality of forming a dough which is elastic enough to hold inside it the gas produced through fermentation and to develop a soft and elastic structure after baking.

High and low molecular weight glutenins, the main storage proteins of the wheat endosperm, are responsible for these particular technological properties. Glutenin's particular sequence enables it to interact in order to form a complex three-dimensional structure that can stretch and trap the carbon dioxide that develops in the leavening phase, thus giving the end product a high specific volume.

Many people are allergic to the gluten contained in wheat flours, in particular to the gliadine and glutenin components with low molecular weight, and thus require particular dietary attentions (Sollid, 2000).

The problem to be solved is to produce non-allergenic flours that nonetheless maintain the property causing them to rise, that is, which are able to form a dough that can be used to make bakery products and that has the same technological properties of dough obtained with wheat flours (Schuppan and Hahn, 2002).

The present invention suggests an optimal solution to this problem and allows non-allergenic, rising flours to be obtained.

### DESCRIPTION

The invention will now be disclosed with reference, solely by way of example, to the technological process of endowing rice flours with the potential to generate a dough that can rise, is elastic and can be used to create bakery products with high specific volumes.

Rice is known to be a cereal with a very peculiar nutritional profile and is considered the most suitable food for children and the elderly.

Rice is in fact a hypoallergenic, highly digestible food, with a protein profile which shows little_diversity but is of very high quality.

Rice's high digestibility is due to the small dimension of its starch granules, which are twenty times smaller-than wheat's and seventy times smaller than the potato's.

Rice is the second cereal after wheat in terms of worldwide production: rice fields cover a hundred and fifty million hectares and produce five hundred million tons of rice each year.

Italy is the main European producer with around two hundred thousand cultivated hectares. Among major cereals, rice has the smallest genome, sixty times smaller than wheat's and twelve times smaller than corn's.

The rice genome, consisting of 12 chromosomes, is completely sequenced. The availability of the sequence of all the genes of this species makes it possible to study its storage protein components and allows its genetic complement to be modified utilizing regions of regulation-specific to the seed storage components.

The invention also concerns the construction of new expression plasmids that allow the production and accumulation—in the seed of cereals such as rice, corn and soybean—of storage proteins of wheat and enzymes of animal origin. The new expression plasmids allow the tissue-specific accumulation of proteins.

In the present invention the design and realization of the expression system in plants is documented, with the demonstration of its validity in a plant such as rice.

In order to obtain the seed-specific expression of proteins, the promoters and signal sequences belonging both to the wheat genes and to the rice storage proteins genes were used.

These regulation and structural sequences were isolated and cloned from the wheat varieties Cheienne, Centauro, Golia, Pandas and Veronese. The gene for the animal enzyme transglutaminase was cloned starting from the cDNA of the liver tissue of a guinea pig. All the cloned gene components were controlled at the sequence level. The cloned sequences were used as such or after mutagenesis to eliminate possible epitopes known as activators of the immune response in patents with gluten allergy.

The final constructs used for rice transformation, also usable in other cereals and in legumes, were realized in vectors of the pUC 19 type and with these, through co-transformation of the constructs with physical methods, immature embryos of Ariete and Rosa Marchetti rice cultivars were transformed. For each transformation experiment, performed using up to ten constructs in various combinations, 100 transgenic (T₀) plant resistant to hygromycin were selected and these were controlled at a molecular level with PCR techniques. The further combination of the genes of interest in a single transgenic line was realized through crossing, followed by diploidization of aploid lines, regenerated by an anthers culture, to reach the homozygosis state faster.

The specificity of accumulation of the various proteins in the seed was controlled with dot blot and Western techniques using polyclonal antibodies developed against the wheat proteins produced in E. coli.

Therefore the invention makes available: (1) new rice varieties characterized by an ability to accumulate different wheat storage proteins and an animal enzyme, also of human origin, able to foster the formation of interchain links between the proteins; (2) new plasmid vectors made for the production of wheat storage proteins in other cereals; (3) a rice flour with technological characteristics similar to the ones of wheat flour.

Another aspect of the invention includes the following components functionally linked by 5′ and 3′ to form a plasmid expression vector: (a) a promoter; (b) a nucleotide sequence corresponding to the aminoacid sequence of the wheat glutenin having a certain c-terminal sequence, or corresponding to guinea pig transglutaminase; c) a signal of polyadenilation.

The DNA sequences from (a) to (c) are cloned in different vectors to form plasmids. The resulting expression plasmids can be used to transform plant cells with direct physical methods. The transformed plant cells are selected and induced to form entire fertile plants that produce seeds able to express the genes of storage or enzymatic proteins.

Another key element of the invention includes nucleotide sequences of wheat glutenins that are modified with techniques of direct mutagenesis in order to eliminate aminoacid sequences known as allergenic in food allergies to gluten.

Another aspect of the invention regards the use of flours taken from seeds of plants transformed with the above mentioned plasmids, for the production of baked products, after kneading and fermentation.

### BRIEF DESCRIPTION OF TABLES AND FIGURES

The characteristics, elements and goals of the invention, briefly described earlier, will become much clearer and more understandable when illustrated with reference to tables and figures that follow. It should be noticed, however, that the examples in the figures show preferential elements of the invention and should not be considered as limiting its scope.

Table 1 (enlarged 1A-1B) shows the aminoacid sequences of the wheat proteins chosen for the expression in rice and the preserved C-terminal motive LKVAKAQQLAAQLPAMCR (position 945-962).

Table 2 shows the nucleotide sequence of the gene for the guinea pig transglutaminase enzyme.

Table 3 shows one of the nucleotide sequences of rice's regulation region used for the seed-specific expression of wheat and guinea pig genes.

Table 4 shows the oligo-nucleotide sequence used for the cloning of some wheat storage proteins genes and the guinea pig transglutaminase enzyme.

Table 5 shows the result of an ELISA test performed on wheat flour, rice flour and the new flour of the line PLT3000R13-7.

FIG. 1 shows the plasmid plGP 2001 obtained by cloning the wheat gene 1Bx7.

FIG. 2 shows the plasmid plGP 2002 obtained by cloning the wheat gene 1By9.

FIG. 3 shows the plasmid plGP 2003 obtained by cloning the wheat gene 1Dx5.

FIG. 4 shows the plasmid plGP 2004 obtained by cloning the wheat gene 1Dy10.

FIG. 5 shows the plasmid plGP 2005 obtained by cloning the wheat gene 1Ax2.

FIG. 6 shows the plasmid plGP 2006 obtained by cloning the wheat gene 1Bx17.

FIG. 7 shows the plasmid plGP 2008 obtained by cloning the wheat gene GluHMW2.

FIG. 8 shows the plasmid plGP 2009 obtained by cloning the wheat gene Glu1A.

FIG. 9 shows the plasmid plGP 2010 obtained by cloning the wheat gene 1Ax1.

FIG. 10 shows the plasmid plGP 2012 obtained by cloning the wheat gene 1Dy12.

FIG. 11 shows the plasmid plGP 2050 obtained by cloning the variant MUT1 of the wheat gene 1Dy10.

FIG. 12 shows the plasmid plGP 2051 obtained by cloning the variant MUT1 of the wheat gene 1By9.

FIG. 13 shows the plasmid plGP 2052 obtained by cloning the variant MUT3 of the wheat gene 1By9.

FIG. 14 shows the plasmid plGP 2100 obtained by cloning the gene that codes for guinea pig's transglutaminase (TG).

FIG. 15 shows, by way of example, an agarose gel with the DNA resulting by amplification through PCR, performed using the specific primers for the single wheat's genes, on DNA extracted by T₀ rice plants transformed with the plasmids of FIGS. 1-14. The agarose gel is colored with Ethidium bromide and photographed under UV light to highlight the amplification products obtained using DNA extracted from leaves of rice lines transformed with the plGP2002 vector and two primers that amplify an internal fragment, about 300 pb, of the gene. M=markers of molecular weight (100 bp ladder Promega); C+=positive control (plasmid DNA); 1-16=single rice plants regenerated on selection medium; 17=negative control (DNA extracted from a plant of the Rosa Marchetti variety). The positive plants are the ones that have the fragment indicated by an arrow.

FIG. 16 shows, by way of example, the results of the Southern analysis performed on some T₁ plants of FIG. 15, transformed with the plasmids of figures 1-14. The Southern analysis is performed using DNA extracted from transgenic lines of rice positive to PCR. As a probe a fragment of the By9 gene was used, the genomic DNA of rice was cut with two enzymes in order to have an indication of the number of copies of the genes present in each line. 1-9=transgenic lines of rice transformed with the pPGI2002 plasmid; C−=negative control (DNA of the Rosa Marchetti variety); C+=positive control.

FIG. 17 shows by way of example a SDS-PAGE gel of total proteins extract from the seeds of the indicated transgenic plants T₂ and their Western analysis, after transfer on membrane, using polyclonal antibodies specific for the wheat storage protein 1By9. The Western analysis is performed on total proteins, extracted from single seeds of transgenic rice, after separation through SDS-PAGE electrophoresis, transfer on membrane and detection in chemiluminescence using as primary antibody a polyclonal produced in rabbit and specific of the protein By9. W=total proteins extracted from the wheat seed; 1-10=total proteins extracted from the seed of transgenic lines of rice in segregation; C+=positive control (protein By9 produced in E.coli).

FIG. 18 shows, by way of example, a SDS-PAGE gel of total proteins extract from the seeds of the indicated transgenic plants and Western analysis of the same, after transfer on membrane, using polyclonal antibodies specific for the guinea pig's transglutaminase (TG). The Western analysis is performed on total proteins, extracted from single seeds of transgenic rice, after separation through SDS-PAGE electrophoresis, transfer on membrane and detection in chemiluminescence using as primary antibody a polyclonal produced in rabbit and specific of the protein transglutaminase (TG). 1-7=total proteins extracted from the seed of some transgenic lines of rice; C+=positive control (protein TG produced in E.coli); C−=negative control (proteins extracted from the Rosa Marchetti variety).

FIG. 19 shows, by way of example, a SDS-PAGE gel of total protein extract from the rice transgenic lines transformed with the gene for the protein 1Dy10 and their Western analysis after transfer on membrane, using a specific polyclonal antibody. The Western analysis is performed on total proteins, extracted from single seeds of transgenic rice, after separation through SDS-PAGE electrophoresis, transfer on membrane and detection in chemiluminescence using as primary antibody a polyclonal produced in rabbit and specific of the Dy10protein. W=total proteins extracted from wheat seed; 1-11=total proteins extracted from seeds of different transgenic lines of rice; C+=positive control (Dy10 protein produced in E.coli); C−=negative control (proteins extracted-from the seed of Rosa Marchetti variety).

FIG. 20 shows, by way of example, a one-dimensional electrophoresis of the storage proteins of some wheat cultivars, in which the bands corresponding to the cloned genes are highlighted. Staining with comassie blu of a SDS-PAGE gel highlights the high molecular weight glutenins in the indicated varieties and used, with other varieties, in the cloning work of the single corresponding genes.

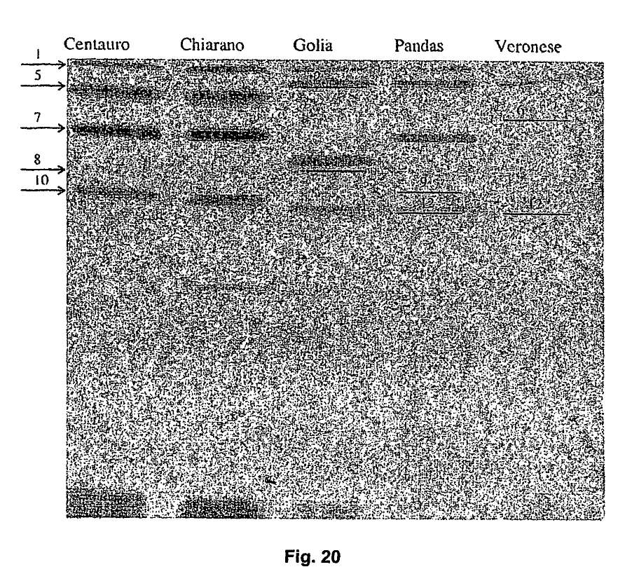

FIG. 21 shows, by way of example, a one-dimensional electrophoresis of the wheat storage proteins where the high molecular weight class and the low molecular weight class of glutenins are visible. Staining with comassie blu of a SDS-PAGE gel highlights the high molecular weight glutenins (higher part of the gel) and the low molecular weight glutenin (lower part of the gel) present in 9 cultivars of bread wheat.

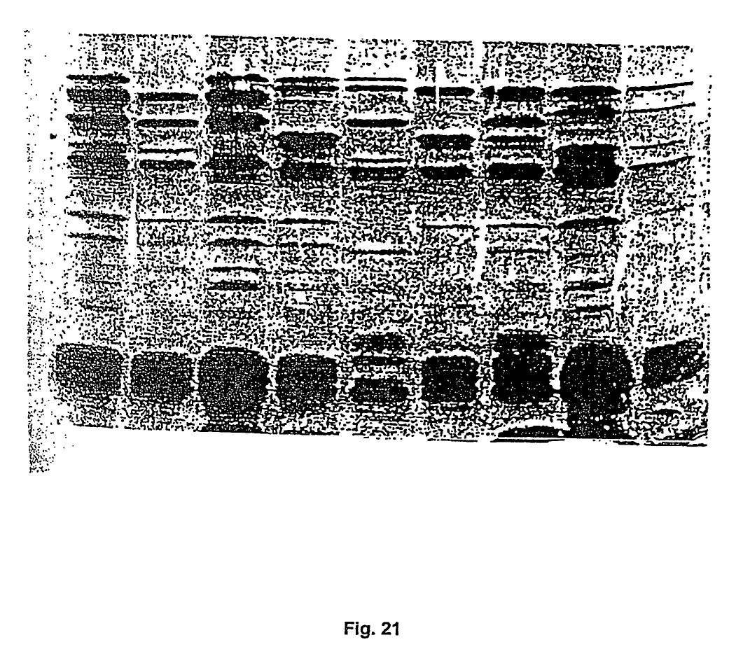

FIG. 22 shows the result of a Western analysis performed on the proteins of FIG. 21, after transfer on membrane, using the serum of a patent with gluten allergy, to highlight the almost exclusive recognition of the low molecular weight glutenins. The Western analysis is performed on total proteins of FIG. 21, after transfer on membrane and detection in chemiluminescence using as primary antibody the IgA+IgG of the serum of a celiac patient.

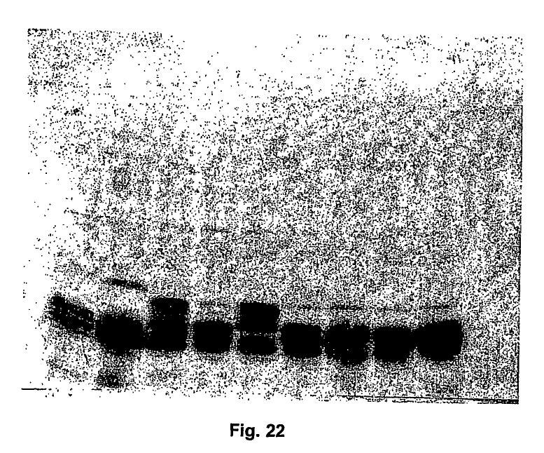

FIG. 23 shows, by way of example, the result of a bread-making test in which the dough-was prepared using flour produced by a transgenic line of rice (on the right) that expresses the wheat proteins 1Ax1, 1Dx2, 1Dx5, 1Bx6, 1Bx7, 1Bx17, MUT11Dx10, MUT11By9 and the enzyme TG, compared with a normal rice flour (on the left).

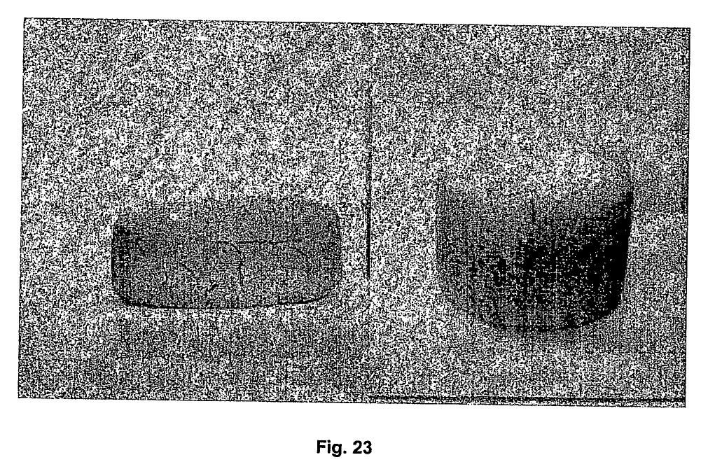

FIG. 24 shows, by way of example, the result of the test described in the FIG. 23 to show the alveolar form and the rising obtained with the new flour compared to a normal rice flour.

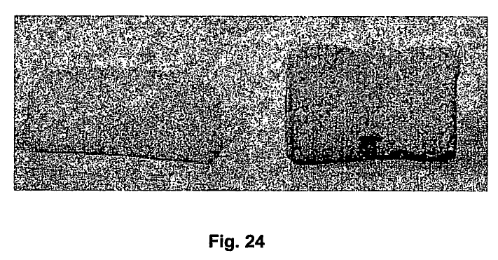

FIG. 25 shows, by way of example, the alveogram obtained with the new flour produced with the seed of the line reported in FIG. 23. It shows also the results of the alveogram performed on the dough obtained from the flour of table 5. The results are P/L=0.78 mm H2O/mm e W=28 E-4J.

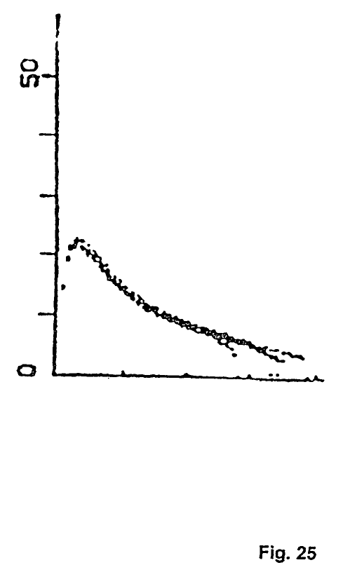

FIG. 26 shows, by way of example, the result of a PCR analysis aimed at demonstrating the presence of the gene for the transglutaminase enzyme in the transformed lines. The agarose gel is stained with Ethidium bromide and photographed under UV light to highlight the amplification products obtained using DNA extracted from leaves of rice lines transformed with the plGP2100 vector and two primers that amplify the gene of about 2070 pb. 1 kb=molecular weight markers; P+=positive control (plasmid DNA); B=negative control (DNA extracted from a plant of the Rosa Marchetti variety). The plants represent the progeny of some transformed lines.

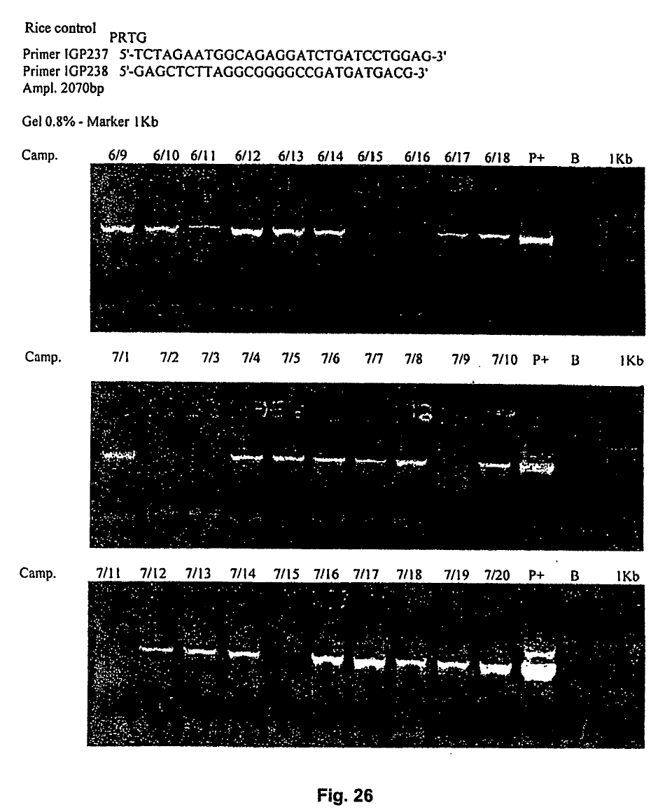

### DETAILED DESCRIPTION OF THE INVENTION

For the cloning of the sequences corresponding to the glutenin genes of high molecular weight, of wheat, with or without the regulation region, the polymerase chain reaction (PCR) technique was used, starting from the information sequences present in the databank. Genomic DNA extract from the leaves of Triticum Aestivum cultivar Cheienne, Chiarano, Centauro, Golia, Pandas and Veronese was used. Some of the oligonucleotides used for the specific amplification are reported in table 4.

For the cloning of the sequence corresponding to the guinea pig's gene that codes for the transglutaminase enzyme, the RT-PCR technique was used. In this case total RNA extract from guinea pig's liver was used and the amplification specific oligonucleotides are reported in table 4.

Once cloned, the genes that code for the wheat proteins were used as such or after site-direct mutagenesis to replace specific aminoacids.

Specifically, the modified nucleotide sequences code for the aminoacid sequences of the type—as a non-restrictive example—PFPQPQLPY, PQPQLPYPQ, PYPQPQLPY, LQLQPFPQPQLPY, QQGYYPTSPQQSG, QQGYYPTS, PFSQQQQQ, QSEQSQQPFQPQ and QXPQQPQQF paying special attention to the replacement of the glutamine and of the other aminoacids in underlined positions (Willemun et al., 2002; Shan et al., 2002).

For the rice promoter PROL we started from sequence information gained from experiments of differential display that highlighted the specificity of expression in the seed of the original clone. After the comparison of the obtained sequence with the databank the clone resulted matching 100% with the sequence with Acc. Number AF156714, and from this we started cloning, using the PCR technique from the genomic DNA of Ariete variety.

The wheat amplification products correspond to the expected dimensions for the specific genes according to the EMBL sequence data.

In the case of the rice promoter, the template DNA was extracted from the leaves of Oryza Sativa cultivar Ariete and the product of the amplification corresponds to the expected dimensions according according to the EMBL sequence data.

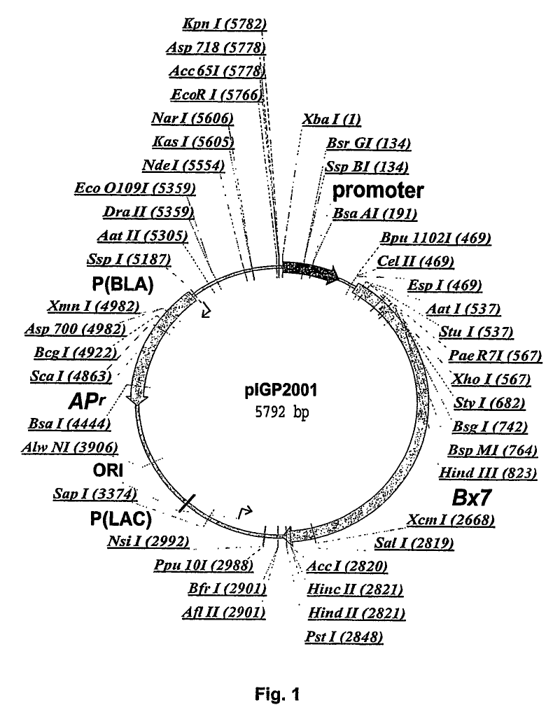

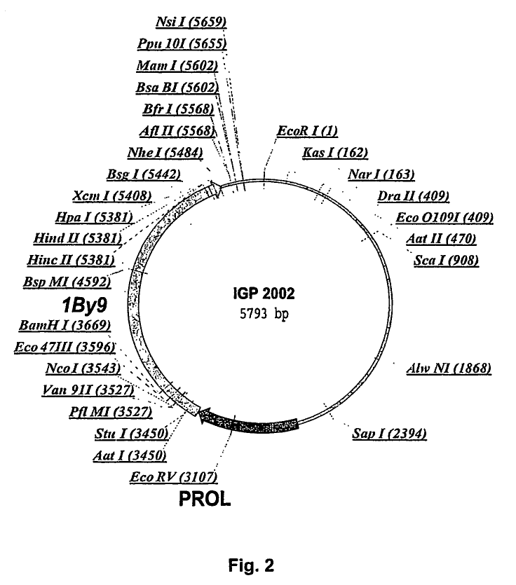

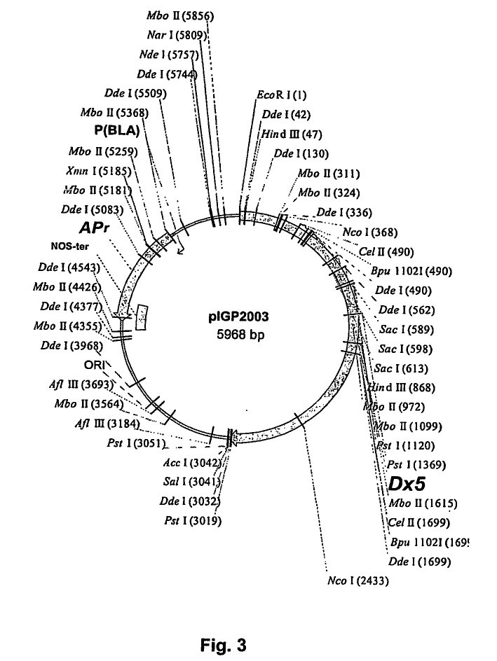

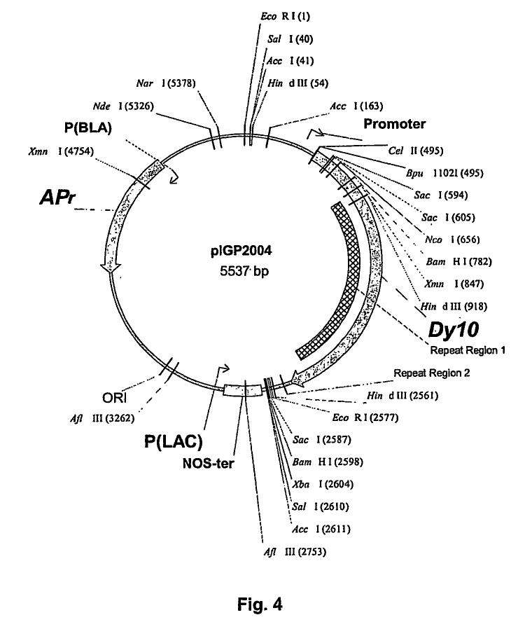

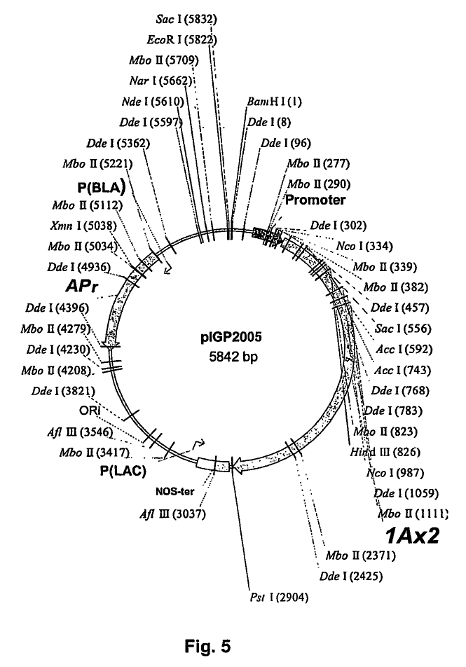

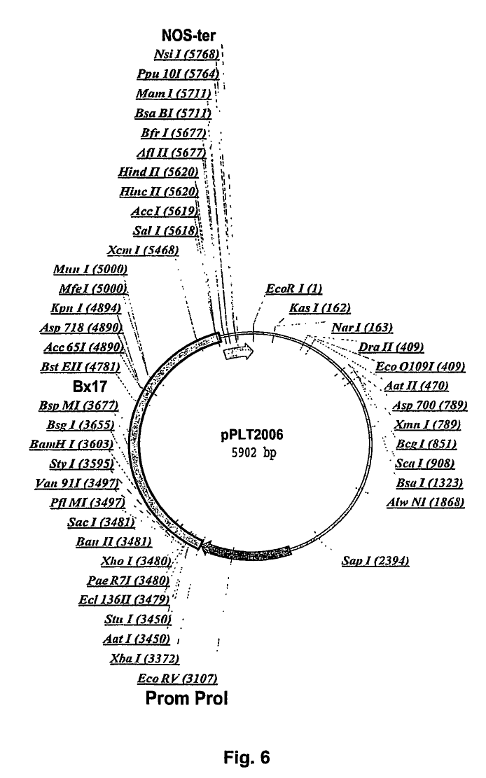

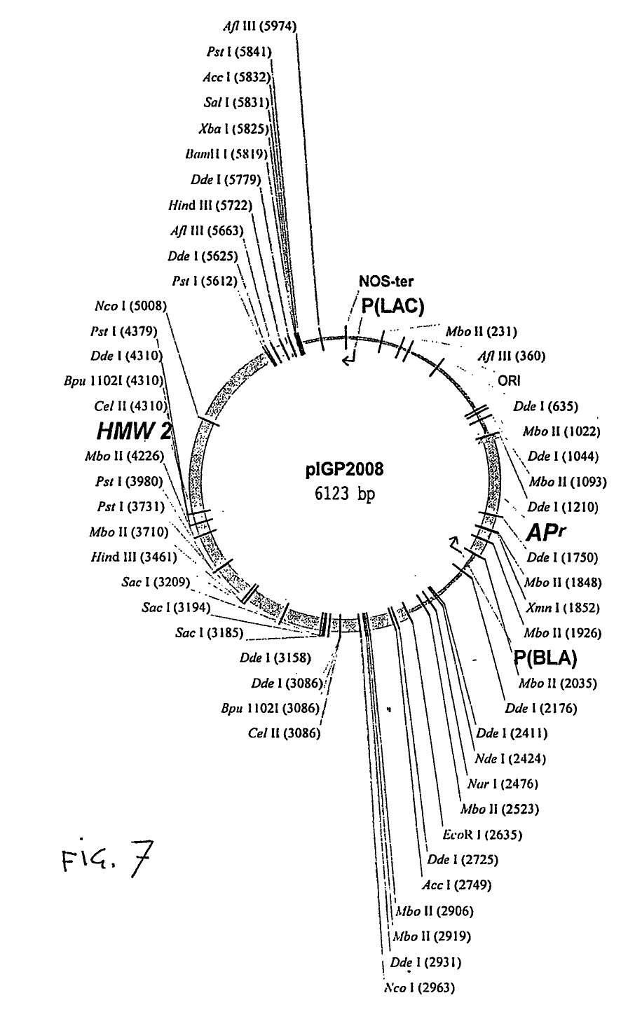

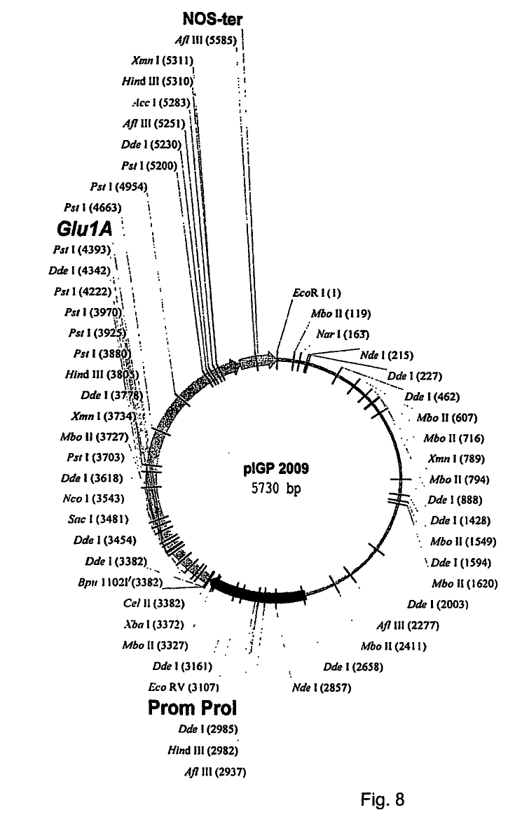

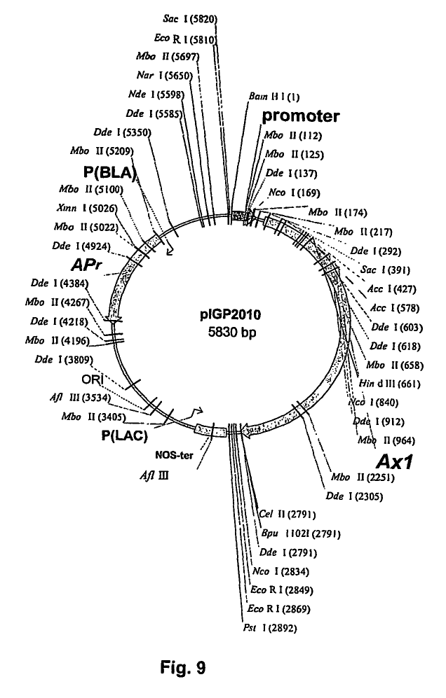

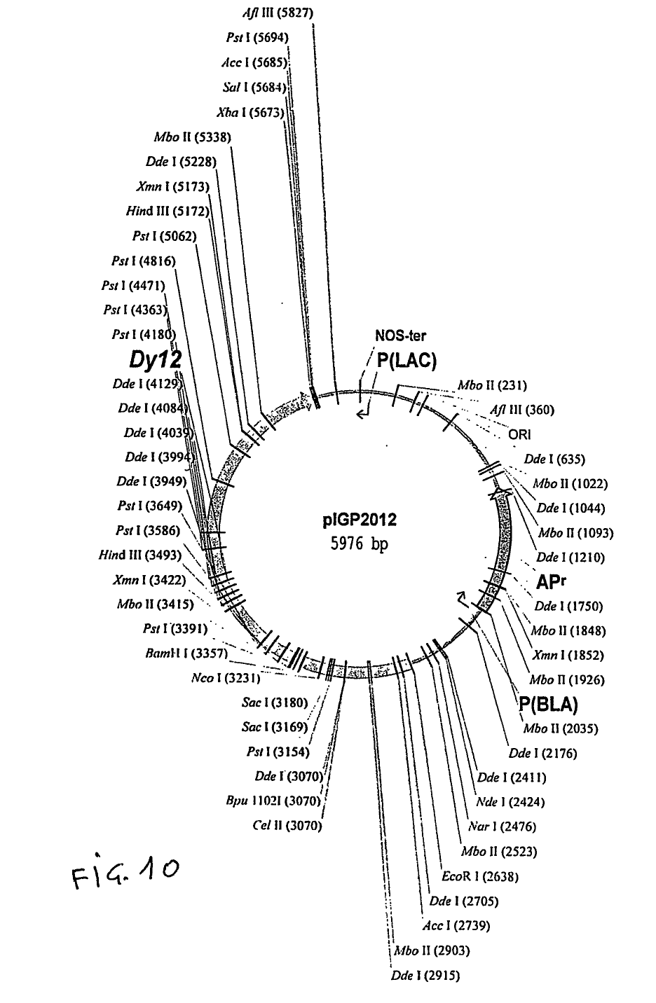

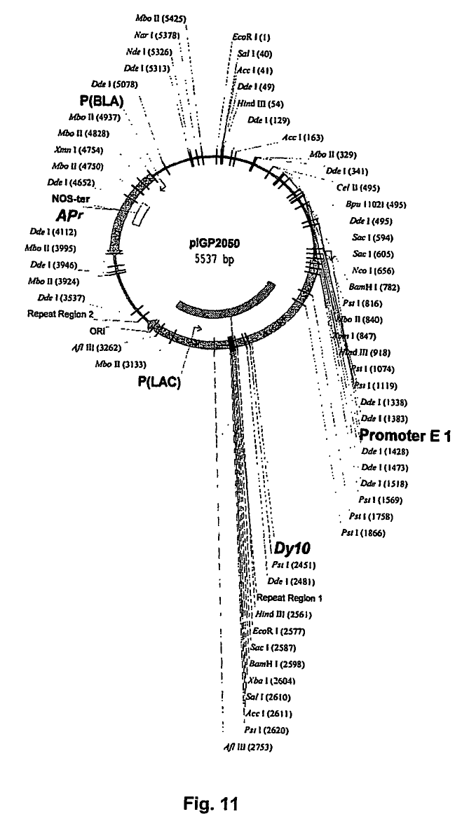

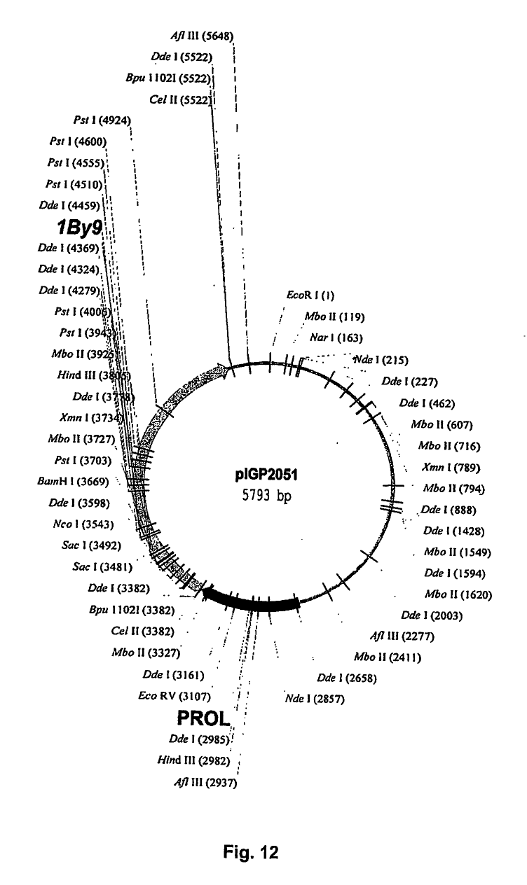

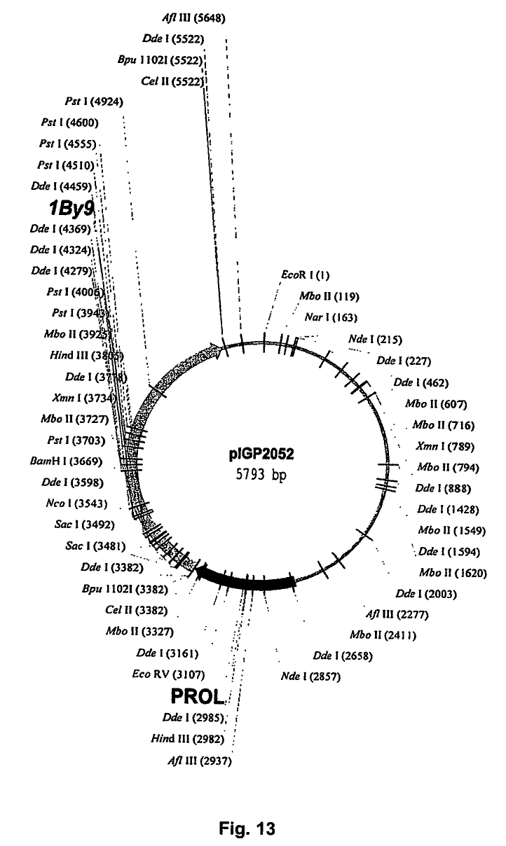

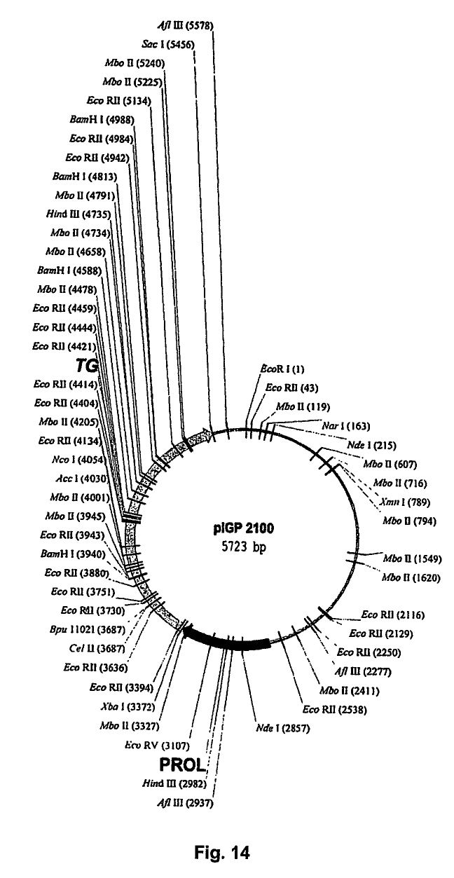

Starting from the amplified fragments, through ligation in the vector pGEM-T, the vectors were built from which the single fragments were recuperated, using the indicated enzymes, to insert them in the vector pPLT 100 (derived from pUC19) to obtain the final constructs shown in FIGS. 1-14.

The final plasmids were verified through restriction analysis using different enzymes and one clone for each type was chosen and sequenced. The sequenced clones turned out to be identical to the sequences present in the databank, with the exception of the sequence of the promoter PROL, which shows some nucleotide differences compared to the sequence in the databank.

The plasmids plGP2001, plGP2002, plGP2003, plGP2004, plGP2005, plGP2006, plGP2008, plGP2009, plGP2010, plGP2012, plGP2050, plGP2051, plGP2052, plGP2100 and plGP2500 (which carries the hygromycin resistance gene used for the selection of the transformed) were purified from cellular cultures of E. coil and the DNA utilized for the transformation of rice embryos with biolistic technique.

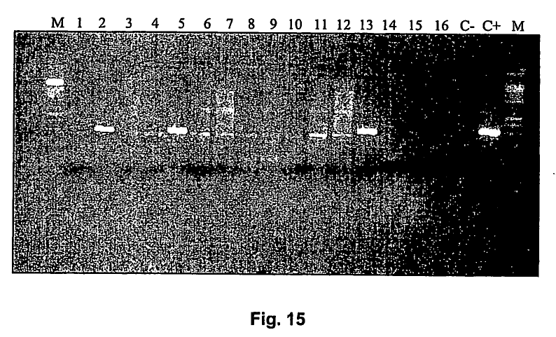

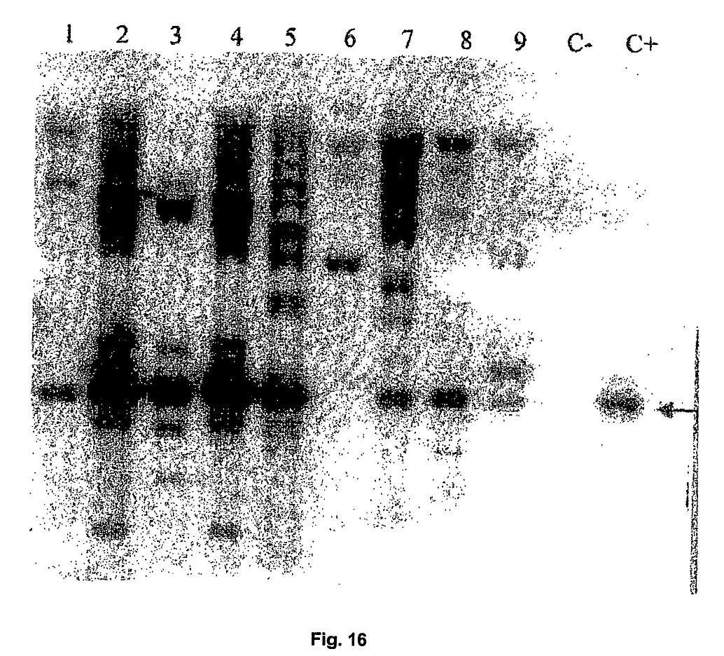

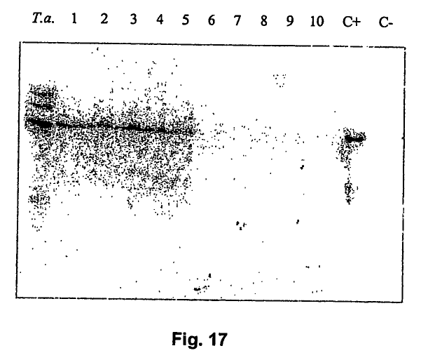

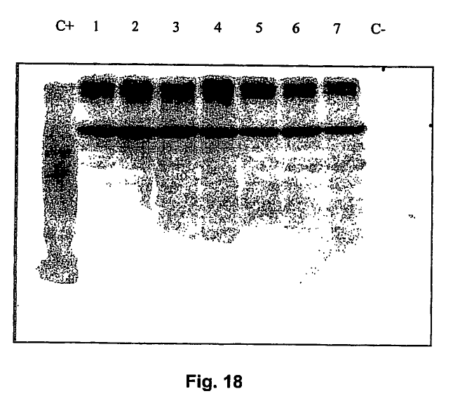

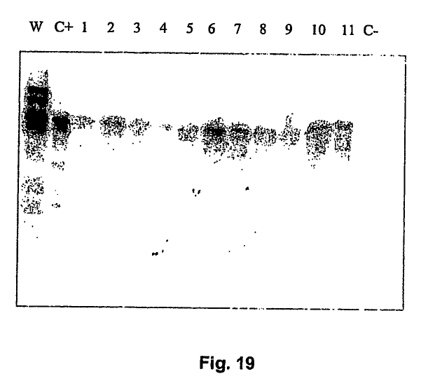

The T₀ plants were verified through PCR analysis (FIG. 15), the T₁ plant through Southern analysis (FIG. 16) and the T₂ plant, and following generations, through Western analysis (FIGS. 17, 18 and 19).

The PCR positive plants show the accumulation of the corresponding protein, recognized by the specific antibody, only in the seed.

The presence of the recombinant protein only in the seed and not in the leaves was verified in all the examined transgenic plants.

### EXAMPLE 1

### Cloning of the Genes that Code for Wheat Proteins

The genes of interest were cloned starting from genomic DNA of wheat extracted from single varieties known as having a good expression of the protein of interest. The bread wheat Cheienne, Chiarano, Centauro, Golia, Pandas and Veronese were mainly used. The genomic DNA was used as the template in PCR reactions that had to be optimized for each single gene (Mullis and Faloona, 1987). As an example, the conditions applied for the amplification of the gene Ax1 are reported here: initial denaturation at 98° C. for three minutes, followed by 38 cycles of denaturation at 95° for one minute, annealing at 62° for one minute, extension at 72° for four minutes, followed by a final synthesis at 72° for ten minutes. The primers used were drawn for each single gene (table 4) considering both the structural part by itself, from the ATG to the stop codon, and the structural part plus the regulation region in 5′ and in 3′.

The amplified fragments were cloned in the vector pGEM-T (Promega), sequenced and subcloned in vectors for expression in E.coli (pET 28a, Novagen) to produce the protein to be used in the immunization of rabbits, and in vectors for specific expression in rice (pPLT 100). In cases in which the genes were modified, they underwent several cycles of mutagenesis performed in the vector pGEM, followed by a further sequencing to verify the variations introduced in the codons.

### EXAMPLE 2

### Genetic Transformation of Rice Embryos

The plants of the rice varieties Ariete and Rosa Marchetti, chosen for the genetic transformation, were seeded in a greenhouse in March.

At flowering, the single spikleets were labeled indicating the exact date of flowering and after 11 days the immature embryos were excised from the seed, in sterile conditions, for genetic transformation with physical methods with the instrument PDS-1000/He (BioRad). The genetic modification was performed using a co-transformation technique where the selection marker (resistance to hygromycin) was present on a plasmid (plGP 2500) separate from those containing the genes of interest (plGP 2001 to 2100).

In the transformation experiments the total concentration of DNA was 1 μg/μl, using 0.6 μg of DNA for each shooting of target tissue. The ratio between the DNA with the selection marker and the DNA with the gene, or genes, of interest was 1:5 (calculated on the number of molecules). When the transformation included several genes of interest the ratio remained constant between the selection plasmid and the plasmids with the gene of interest (1:5), while the genes of interest remained in a 1:1 ratio with one another (e.g. for 6 genes the final molar ratio was 1:5:5:5:5:5:5). The transformation was performed transferring the marker plasmid in combination with a single plasmid or with several plasmids (up to 10) with the genes of interest (Chen et al., 1998).

In the case of transformation with one or few genes of interest, or when the molecular analysis highlighted the presence of only some of the introduced genes, the transgenic lines obtained were crossed to combine different genes in a single line. The segregating plants, which displayed the genes of interest, were diploidized starting from haploids regenerated from anthers cultures, to reach the homozygosis status for all the single genes.

The target explants, roughly 30 immature embryos, were gathered six days after sampling at the center of a Petri dish containing the osmotic medium NB—with 0.4 M mannitol. After incubation for four hours the embryos were shot twice, using gold particles with a 1.5-3.0 micron diameter, at a pressure of 1100 psi and 27 in Hg vacuum.

Twenty four hours after the shooting the embryos were individually transferred into an NB medium and incubated for three days at 28° C. in the dark, then transferred to a solid selection medium containing 50 mg/liter of hygromycin B. After two weeks of selection the embryos were transferred to an R2 liquid selection medium (Ohira et al. 1973) supplemented with 1 mg/l of 2,4-D, 1 mg/l thiamine, 30 g/l saccharose and 50 mg/l hygromycin B. Embryos were maintained at 90 rpm on a rotating plate for another two weeks; the medium was changed in the middle of said period. When the hygromycin-resistant calluses became visible, they were transferred to a medium to increase the callus mass (R21) and afterwards to a regeneration medium (MS) containing 2.5 mg/l BAP and 0.5 mg/l NAA, exposed to light, with a 16-hour photoperiod. The regenerated shoots were then transferred to a radication medium for four weeks and afterwards to pots in the greenhouse.

### EXAMPLE 3

### Production of Dough

Dough was prepared using the same procedure for wheat flour (Veronese variety), rice flour (Rosa Marchetti variety) and the new flour (transgenic line PLT300R13-7). 500 grams of flour were mixed with 350 ml of water, 10 grams of salt, 10 grams of sugar and 7 grams of dry active yeast. The dough was obtained using an autobakery and kneading the mixture for a 10-minute period. The dough was kept rising for one hour at 37° C., followed by cooking at 200° for 60 minutes.

### BIBLIOGRAPHY

Chen L., et al. 1998. Nature Biotechnology 16: 1060

Mullis K. B., Faloona F. A. 1987. Method. Enzymol. 155: 335.

Ohira K. Ojima K., Figiwara A. 1973. Plant Cell Physiol. 14:1113.

Sanford J. C., Smith F. D., Russel J. A. 1993. Meth. Enzymol. 317:483.

Schuppan D., Hahn E. G. 2002. Science 297:2218.

Shan L. et al. 2002. Science 297:2275.

Sollid L. M. 2000. Annu. Rev. Immunol. 18:53

Willemun V., et al. 200. Gastroenterology 122:1729.

TABLE 1a

| | |
|---|---|
| | 1 10 20 30 40 50 60 70 80 90 |
| | \|--------+---------+---------+---------+---------+---------+---------+---------+---------+ |
| Ax1 | MTKRLVLFAAVVVALVALTAACGEASGQLQCCRELQEHS---LKACRQVVDQQL------------------RDVSPECQPVGGGPVARQ |
| Ax2 | MTKRIVIFAAVVVAIVAITAAFGFASGQIQCFRFIQEHS---LKHCRQVVDQQI------------------RHVSPECQPVGGGPVARQ |
| Dx5 | MAKRLVLFVAVVVALVALTVACGCASEQLQCCRELQELQERELKACQQVHDQQL------------------RDISPECHPVVVSPVAGQ |
| HMW2(X03346) | MAKRIVIFVHVVVHIVAITVAFGFASEQIQCFRFIQELQERELKRCQQVHDQQI------------------RDISPECHPVVVSPVRGQ |
| Dx7 | MAKRIVLFAAVVVALVALTAACGCASGQLQCCKCLE--------ACQQVVDQQL------------------RDVSPGCRPITVSPGTRQ |
| Dy12 | MAKRIVIFAAVVIAIVAIITAEGFASRQIQCFRIIQESS---LERCRQVVDQQIAGRLPWSTGLQMRCCQQLRDVSAKCRSVAVSQVAKQ |
| Dy10 | MAKRLVLTQQVVIDLVALITACGCASRQLQCCRCLQESS---LEHCRQVVDQQLAGRLPWSTGLQMRCCQQLKDVSAKCRSVAVSQVARQ |
| Dy9 | MAKRIVIFATVVITIVAIIAHLGFHSRQLQCIRFIQFSS---LEHCRQVVDQQIAGRLPWSTGLQMRCCQQLRDVSAKCRPVAVSQVVRQ |
| glu10(X03042) | MAKRLVLCOTVVIGLVSLTVACGERSKQLQCERELQESS---LEHCRLVVDQQLASRLPWSTGLQMRCCQQLRDISAKCRPVALSQVARQ |
| Consensus | MaKRLVLFaaVVvaLVaLl.ALGEHS.QLQCErECyo.u...l.HCryVvDQQL..................RDvSp.Crpv.vcpvarQ |

| | |
|---|---|
| | 100 110 120 130 |
| | ---------+---------+---------+---------\| |
| Ax1 | YEQQVVVPPKGGSTYPGETTPPQQLQQSILWGIPALLRR- |
| Ax2 | YEQQVVVPPKGGSFYPGFTTPPQQIQQSILWGIPALLRR- |
| Dx5 | YEQQIVVPPKGGSFYPGETTPPQQLQQRIFWGIPALLKR- |
| HMW2(X03346) | YEQQIVVP-KGGSFYPGFTTPPQQIQQRIFWGIPALIKR- |
| Dx7 | YEQQPVVPSKAGSFYPSETTPSQQLQQMIFWGIPALLRR- |
| Dy12 | YCQT-VVPPKGGSFYPGFIIPLQQIQQGIFRGTSSQTVQG |
| Dy10 | YCQT-VVPPKGGSFYPGETTPLQQLQQGIFWGTSSQTVQG |
| Dy9 | YFQT-VVPPKGGSFYPGFTTPLQQIQQVIFWGTSSQTVQG |
| glu10(X03042) | YGQT-AVPPKGGPFYHRCTTPLQQLQQGIFGGTSSQTVQG |
| Consensus | YeQq.vVPpKgGvtYpgEIIP.QQLQQ.IfuGlpall.r. |

| | |
|---|---|
| | 131 140 150 160 170 180 190 200 210 220 |
| | \|--------+---------+---------+---------+---------+---------+---------+---------+---------+ |
| Ax1 | YYLSVISPQQVSYYPGQASSQRPGQGQQP------GQGQQE---------------YYLTSPQQSGDWQQPGQGQAGYYPISPQQSGQEQ |
| Ax2 | YYLSVTSPQQVSYYPGQASSQRPGQGQQ------------E---------------YYLTSPQQSGDWQQPGQGQSGYYPTSPQQSGQKQ |
| Dx5 | YYPSVTCPQQVSYYPGQASPQRPGQGQQP------GQGQQ---------------GYYPTSPQQPGQWEEPEQGQQGYYPISPQQPGQLQ |
| HMW2(X03346) | YYPSVTSPQQVSYYPGQASPQRPGQGQQP------GQGQQSGQGQQ---------GYYPTSPQQPGQWQQPEQGQPGYYPTSPQQPGQLQ |
| Dx7 | YYPSVTSSQQGSYYPGQASPQQSGQGQQP------GQEQQPGQGQQDQQPGQRQQGYYPTSPQQPGQGQQLGQGQPGYYPTSQQPGQKQQ |
| Dy12 | YYPSVTSPRQGSYYPGQASPQQPGQGQQPGKWQEPGQGQQWYYPTSL---------------QPPGQGQQIGKGKQGYYPTSLQQPG--- |
| Dy10 | YYPGVTSPRQGSYYPGQQSPQQPGQGQQPGKWQEPGQGQQWYYPTSL---------------QQPGQGQQIGKGQQGYYPTSLQQPG--- |
| Dy9 | YYPSVSSPQQGPYYPGQASPQQPGQGQQPGKWQELGQGQQGYYPTSLHQSGQGQQGYYPSSLQQPGQGQQIGQGQQGYYPTSLQQPG--- |
| glu10(X03042) | YYPSVISPQQGSYYPGQASPQQPG------KWQCLGQGQQWYYPTSLQQPGQGQQGYYRTSLQQPGQRQQ------GYYRTSLQQPG--- |
| Consensus | YYpuVtapqQguYYPGQASpQypGqgyqp......vqvqy...............cyy.tupQQpGQ.qy.gqvq.GYYpIS.Qqpy..q |

| | |
|---|---|
| | 230 240 250 260 |
| | ---------+---------+---------+---------\| |
| Ax1 | PGYYPTSPWQPEQLQQPTQGQQRQQPGQGQQLRQGQQGQQ |
| Ax2 | PGYYPTSPWQPEQLQQPTQGQQRQQPGQGQQLRQGQQGQQ |
| Dx5 | ------------------------QPAQGQQPQQGQQGQQ |
| HMW2(X03346) | ------------------------QPRQGQQPGQQQGGRQ |
| Dx7 | ---------------QGQQSGQGQQGYYPTSPQQSGQGQQ |
| Dy12 | ------------------------------------QGQQ |
| Dy10 | ------------------------------------QGQQ |
| Dy9 | ------------------------------------QGQQ |
| glu10(X03042) | ------------------------------------QGQQ |
| Consensus | ........................q........q..QGqQ |

| | |
|---|---|
| | 261 270 280 290 300 310 320 330 340 350 |
| | \|--------+---------+---------+---------+---------+---------+---------+---------+---------+ |
| Ax1 | SGQGQPRYYPISSQ-QPGQLQQLAQGQQGQQPERGQQGQQSGQGQQLGQGQQGQQPGQKQQSGQGQQGYYPISPQQLGQG---QQSGVGQ |
| Ax2 | SGQGQPRYYPTSSQ-QPGQLQQLAQGQQGQQPERGQQGQQSGQGQQLGQGQQGQQPGQKQQSGQGQQGYYPISPQQLGQG---QQSGQGQ |
| Dx5 | PGQGQPGYYPISSQLQPGQLQQPAQGQQGQQPGQRQQGQQPGQGQQPGQGQQGQQPGQGQQPGQGQQGQQLGQGQQGYYPTSLQQSGQGQ |
| HMW2(X03346) | PGQGQPGYYPTSSQLQPGQLQQPAQGQQGQQPGQGQQGQQPGQGQQPGQGQQGQQPGQGQQPGQGQQGQQLGQGQQGYYPTSLQQSGQGQ |
| Dx7 | PGQGQPGYYPISPQ-------------QSGQWQQPGQGQQPGQGQQSGQGQQGQQPGURPGQGQQGYYPISPQQPGQG------------ |
| Dy12 | TGQGQQGYYPISPQH-TGQRQQPVQGQQIGQGQQPEQG------QQPGQWQQGYYPTSPQQLGQGQQ----------------------- |
| Dy10 | ------GYYPISLQH-TGQRQQPVQGQQ------PEQG------QQPGQWQQGYYPTSPQQLGQGQQ----------------------- |
| Dy9 | TGQGQQGYYPISPQH-PGQRQQPGQGQQTGQGQQLGQGRQTGQGQQSGQGQQGYYPTSPQQLGQGQQ----------------------- |
| glu10(X03042) | IGQWQQGYYPISPQH-PGQGQQPGQVQKIGQGQQPEKGQQLGQGQQIGQGQQPE---------QGQQ----------------------- |
| Consensus | .gqwqpyYYPIS.Q..pgq.qyp.qgqq..q..q..qliqq.gqgQQ.GQgQQgqqpgq.qq.gQGQQg......qq............. |

| | |
|---|---|
| | 360 370 380 390 |
| | ---------+---------+---------+---------\| |
| Ax1 | LGYYPTSPQQSGQGQSGYYPISRQQPGQLQQSTQEQQLGQ |
| Ax2 | LGYYPTSPQQSGQGQSGYYPTSAQQPGQLQQSTQEQQLGQ |
| Dx5 | PGYYPTSLQQLGQGQSGYYPISPQQPGQGQQPGQLQQPRQ |
| HMW2(X03346) | PGYYPTSLQQLGQGQSGYYPISPQQPGQGQQPGQLQQPRQ |
| Dx7 | --------QQSGQGQPGYYPISLRQPGQWQQPGQ------ |
| Dy12 | ----PGQWQQSGQGQQGHYPTSLQQPGQGQQGHYLASQQQ |
| Dy10 | ----PRQWQQSGQGQQGHYPISLQQPGQGQQGHYLASQQQ |
| Dy9 | ----PGQWQQSGQGQQGYYPISQQQPGQGQQGQYPASQQQ |
| glu10(X03042) | ----PGQGQQPGQGQQGYYPISLQQPGQGQQ--------- |
| Consensus | ....p...QQnGQGQ.GyYPIS.qQPGQyQQ..q.....q |

| | |
|---|---|
| | 391 400 410 420 430 440 450 460 470 480 |
| | \|--------+---------+---------+---------+---------+---------+---------+---------+---------+ |
| Ax1 | PQQDQQSGQGRQGQQSGQRQQDQQSGQGQQPGQRQPGYYSISPQQLGQGQPRYYPTSPQQPGGFQQPRQLQQPFQGQQGQQPFQGQQGQQ |
| Ax2 | GQQDQQSGQGRQGQQSGQRQQDQQSGQGQQPGQRQPGYYSISPQQLGQGQPRYYPTSPQQPGQEQQPRQLQQPEQGQQGQQPEQGQQGQQ |
| Dx5 | GQQP---GQGQQGQQPGQGQQGQQPGQGQQPGQGQPGYYPISPQQSGQGQPGYYPISSQQPIQSQQPGQ------------------GQQ |
| HMW2(X03346) | GQQP---CQGQQGQQPGQGQQGQQPGQGQQPGQGQPGYYPTSPQQSGQGQPGYYPTSSQQPTQSQQPGQ------------------GQQ |
| Dx7 | ------------GQQPGQGQQGQQPGQGQQSGQGQQGYYPISLQPGQGQQLGQG-----------QPGYYPTSQQSEQGQQPGQGKQPGQ |
| Dy12 | PGQGQQGQYPASQQQPGQGQQGHYPASQQQPGQGQQGHYPASQQEPGQGQQGQIPASQQQPGQ--------------------------- |
| Dy10 | PGQGQQGHYPASQQQPGQGQQGHYPHSQQQPGQGQQGHYPASQQEPGQGQQGQIPHSQQQPGQ--------------------------- |
| Dy9 | PGQGQQGQYPASQQQPGQGQQGQYPASQQQPGQGQQGHYLASQQQPGQGQQRHYPASLQQPGQ--------------------------- |
| glu10(X03042) | ------------------------PGQRQQPGQGQQHYYPISLQQPVQGQQGHYPASQHQPGQ--------------------------- |
| Consensus | ..q.qq......yqqpgqgqquyqpgqgQQpGQgQqGyYpIS.QqpgQGQqg.yp.a.qqpgq..qp......................q |

| | |
|---|---|
| | 490 500 510 520 |
| | ---------+---------+---------+---------\| |
| Ax1 | PGQGFQGQQPGQGQQGQQPGQGQPGYYPISPQQSGQGQP- |
| Ax2 | QRQGEQGQQPGQGQQGQQPGQGQPGYYPISPQQSGQGQP- |
| Dx5 | GQQYGQGQQAQQPGQGQQPGQGQPGYYPISPQQSGQGQP- |
| HMW2(X03346) | GQQYGQGQQAQQPGQGQQPGQGQPGYYPTSPLQSGQGQP- |
| Dx7 | GQQGYYPTSPQQSGQGQQLGQGQPGYYPISPQQSGQGQQS |
| Dy12 | ------GQQQHYPASLQQPGQ--QGHYPISLQQLGQGQ-- |
| Dy10 | ------GQQGHYPASLQQPGQGQQGHYPISLQQLGQGQ-- |
| Dy9 | ------GQQGHYTASLQQPGQGQQGHYPASLQQVGQGQ-- |
| glu10(X03042) | ------GQQGHQPASLQKSGQGQQGHYPASLQQPGQGK-- |
| Consensus | ..q...gqq..qp.qgQqpGQgqpGyyPtSpqQoGQGq.. |

| | |
|---|---|
| | 521 530 540 550 560 570 580 590 600 610 |
| | \|--------+---------+---------+---------+---------+---------+---------+---------+---------+ |
| Ax1 | -----GYYPISPQQSGQLQQPGQGQQPQQGQQGQQPGQGQQGQQPGQGQQPGQGQ---------PGYYPISPQQSGQFQQLEQWQQSGQG |
| Ax2 | -----GYYPTSPQQSGQLQQPRQGQQPGQEQQGQQPGQGQQ---------PGQGQ---------PGYYPTSPQQSGQCQQLCQWQQSGQG |
| Dx5 | -----GYYLTSPQQSGQGQQPGQLQQSAQGQKGQQPGQGQQPGQGQQGQQPGQGQQGQQPGQGQPGYYPTSPQQSGQGQQPGQWQQPGQG |
| HMW2(X03346) | -----GYYLISPQQSGQGQQPGQLQQSAQGQKGQQPGQGQQPGQGQQGQQPGQGQQGQQPGQGQPGYYPTSPQQSGQGQQPGQWQQPGQG |
| Dx7 | GQGQQGYYPISPQQSGWGQQPGQQQSGYFPTSRQQSGQGQQPGQ---GQQSGQGQQGQQPGQGQQRYYPISSQQSRQRQQAGWQQRPGQG |
| Dy12 | -------------QIGQPGQKQQPGQGQQTGQGQQPCQCQQPGQGQQGYYPTSL------------------------QQPGQGQQQGQG |
| Dy10 | -------------QIGQPGQKQQPGQGQQTGQGQQPLQEQQPGQGQQGYYPISL------------------------QQPGQGQQQGQG |
| Dy9 | -------------QIGQLGQRQQPGQGQQIRQGQQLCQGQQPGQGQQTRQGQQLCQGQQPGQGQQGYYPTSPQQSGQGQQPGQSQQPGQG |
| glu10(X03042) | -------------QIGQRLQRQQPGQGQQIGQGQQPEQEQQPGQGQQGYYPIYL------------------QQPGQGQQPEQRQQPGQG |
| Consensus | ...gyy.t.apqQaGQ.qQp.Q.qqg.q..qgQQpgQgQQpgqgqqq.qpgqgq...........gyyptspqqsgq.QQpgQwQqpGQG |

| | |
|---|---|
| | 620 630 640 650 |
| | ---------+---------+---------+---------\| |
| Ax1 | QPGHYPTSPLQPGQGQPQ------------YYPISPQQTG |
| Ax2 | QPGHYPISPLQPGQGQPG------------YYPTSPQQIG |
| Dx5 | QPGYYPTSPLQPGQGQPG------------YQPTSPQQPG |
| HMW2(X03346) | QPGYYPISPLQPGQGQPG------------YQPTSPQQPG |
| Dx7 | QPGYYPISPQQPLQLQQSGQAQQSGQWQLVYYPISPQQPG |
| Dy12 | QQGYYPTSLQQPGQGQQGH------------YTASLQQPG |
| Dy10 | QQGYYPISLQQPGQGQQGH------------YTASLQQPG |
| Dy9 | QQGYYSSSLQQPGQGLQGH------------YPASLQQPG |
| glu10(X03042) | QQGHYPQSLQQSGQGQQGH------------YPASLQQLG |
| Consensus | QpGgYptSpqQpGQgqqg............unprSpQQpg |

| | |
|---|---|
| | 651 660 670 680 690 700 710 720 730 740 |
| | \|--------+---------+---------+---------+---------+---------+---------+---------+---------+ |
| Ax1 | QGQQPGQLQQPTQGQQGQQ------------------------------------------------------PGQGQQGQQPGQGQQGQ |
| Ax2 | QGQQPGQLQQPTQGQQGQQ------------------------------------------------------PGQGQQGQQPGEGQQGQ |
| Dx5 | QGQQPGQLQQPRQGQQGQQLAQGQQGQQPRQYQQGQQPRQGQQGQQLGQGQQGQQPGQGQQGQQPRQGQQGQQPGQGGHGQQPGQGQQGQ |
| HMW2(X03346) | QGQQPGQLQQPRQGQQGQQLRQGQQGQQPRQVQQGQQPRQGQQGQQLGQGQQGQQPGQGQQ------------PRQGQQGQQPGQGQQGQ |
| Dx7 | QGQQPRQGQQPRQGQQSAQEQQPGQRQ------------------------------------------------QSGQWQLVYYPTSPQ |
| Dy12 | QGQ--------------------------------------------------------------------------GQPGQRQQPGQGQ |
| Dy10 | QGQ----------------------------------------------------------------------------PGQRQQPGQGQ |
| Dy9 | QGQ----------------------------------------------------------------------------PGQRQQPGQGQ |
| glu10(X03042) | QGQ----------------------------------------------------------------------------PGQTQQPGQGQ |
| Consensus | Qgqpq.q.qqp.qgqq..q........................................................q..q.qq..qp.qgQ |

| | |
|---|---|
| | 750 760 770 780 |
| | ---------+---------+---------+---------\| |
| Ax1 | QPGQGQQPGQGQPGYYPISLQQSGQGQQPGQWQQPGQ--- |
| Ax2 | QPGQGQQPGQGQPGYYPISLQQSGQGQQPGQWQQPGQ--- |
| Dx5 | QPGQGQQPGQGQPWYYPISPQFSGQGQQPGQWQQPGQ--- |
| HMW2(X03346) | QPGQGQQPGQGQPWYYPTSPQESGQGQQPGQWQQPGQWQQ |
| Dx7 | QPGQLQQPGQGQQGYYPISPQQSGQGQQ------------ |
| Dy12 | IPCQGQQPGQGQQGYYPISPQQPGQGQQLGQ--------- |
| Dy10 | HPEQGKQPGQGQQGYYPISPQQPGQGQQLGQ--------- |
| Dy9 | QPCQGQQPGQGQQGYYPISPQQPGQGKQLGQ--------- |
| glu10(X03042) | QPEQEEQSGQGQQGYYPISPQQPGQGQQGHF--------- |
| Consensus | qPgQgqQpgQGQqgYYPISpQqgGQGqQ.gq......... |

| | |
|---|---|
| | 781 790 800 810 820 830 840 850 860 870 |
| | \|--------+---------+---------+---------+---------+---------+---------+---------+---------+ |
| Ax1 | ---GLPGYYPTSSLQPEQGQQGYYPTSQQQPGQGPQPGQWQQSGQGQQGYYPTSPQQSGQGQQPGQWLQPGQWLQSGYYLTSPQQLGQGQ |
| Ax2 | ---GQPGYYPISSLQPEDGQQGYYPISQQQPGQGPQPGQWQQSGQGQQGYYPTSPQQSGQGQQPGQWLQPGQWLQSGYYLISPQQLGQGQ |
| Dx5 | ---GQPGYYLTFSYQARTGQQGYYPTSLQQPGQGQQPGQWQQSGQGQHWYYPTSPKLSGQGQRPGQWLQPGQGQQ-GYYPTSPQQPPQGQ |
| HMW2(X03346) | PGQGQPGYYLISPLQLGQGQQGYYPISLQQPGQGQQPGQWQQSGQGQHGYYPTSPQLSGQGQRPGQWLQPGQGQQ-GYYPTSPQQSGQGQ |
| Dx7 | ------GYYPTSPQQSGQGQQGYYPTSPQQSGQGQQPGQGQQPRQGQQGYYPISPQQSGQGQQPGQGQQ-------GYYPTSPQQSGQGQ |
| Dy12 | ------------------GQQGYYPTSPQQPGQGQQPGQGQQ----------------------------------GHCPWSPQQTGQRQ |
| Dy10 | ------------------GQQGYYPTSPQQPGQGQQPGQGQQ----------------------------------GHCPTSPQQSGQAQ |
| Dy9 | ------------------GQQGYYPTSPQQPGQGQQPGQGQQ----------------------------------GHCPTSPQQTGQRQ |
| glu10(X03042) | ------------------PT-----------------------------------------------------------------SGQRQ |
| Consensus | ......gyy.t.......gqqgyypts.qqpgqgqqpgq.qq..qgq..yyp.sp..sgqgq.pgq..q.......gyyptspqq.gQgQ |

| | |
|---|---|
| | 880 890 900 910 |
| | ---------+---------+---------+---------\| |
| Ax1 | QPR------QWLQPRQGQQGYYPTSPQQSGQGQQLGQGQQ |
| Ax2 | QPR------QWLQPRQGQQGYYPTSPQQSGQGQQLGQGQQ |
| Dx5 | QLG------QWLQPGQGQQGYYPTSLQQTGQGQQSGQGQQ |
| HMW2(X03346) | QLG------QWLQPGQGQQGYYPTSLQQTGQGQQSGQGQQ |
| Dx7 | QPGHEQQPGQWLQPGQGQQGYYPTSSQQSGQGWQSGQGQQ |
| Dy12 | QLGQGQQIGQYQQPGQGQQGYYPTSLQQPGQGQQSGQGQQ |
| Dy10 | QPGQGQQIGQYQQPGQGQQGYYPTSYQQPGQGQQSGQGQQ |
| Dy9 | QPGQGQQIGQYQQPGQGQQGYYPISLQQSGQGQQSGQGQQ |
| glu10(X03042) | QPGQGQQIGQRQQLGQGQQGYYPTSLQQPGQEQQSGQGQQ |
| Consensus | Qpg..qq.gQalQpgQGQQGYYPtSlQQ.GQgqQsGQGQQ |

| | |
|---|---|
| | 911 920 930 940 950 960 970 980 990 1000 |
| | \|--------+---------+---------+---------+---------+---------+---------+---------+---------+ |
| Ax1 | ---GYYPTSPQQSGQGQQGYDSPYHVSQLHQQHSLKVAKAQQLRAQLPAMCRLFGGAALLASQ |
| Ax2 | ---GYYPTSPQQSGQGQQGYDSPYHVSALHQRRSLKYAKAQQLRRQLPAMCRLLGGDALLASQ |
| Dx5 | ---GYY---------------SSYHVSVLHQRASLKVAKAQQLARQLPAMCRLCGGHHLSASQ |
| HMW2(X03346) | ---GYY---------------SSYHVSVFHQRASLKVHKRQQLRRQLPHMCRLEGGDALSASQ |
| Dx7 | ---GYYPTSLWQPGQGQQGYDSPYQVSACYQRARLKVQKRQQLRRQLPAMCRLCGSDALSTRQ |
| Dy12 | SGQGHQPGQGQQSGQEKQGYDSPYHVSALQQAASPMVHKRQQPRTQLPTVCRMLGGDHLSASQ |
| Dy10 | SGQGHQPGQGQQSGQEQQGYDSPYHVSAFQQRRSPMVRKRQQPRTQLPTVCRMFGGRRLSASQ |
| Dy9 | SGQGHQLGQGQQSGQEQQGYDNPYHVNTEQQTASPKVRKVQQPHTQLPIMCRMLGGDALSASQ |
| glu10(X03042) | LGQGHQPGQGQQSGQEQQGYDSPYRVSVFQQRRSPKVAKARRPVAQLPTMCQMLGGDALSHSQNSLQLAWCLGMHAPLSNKRDVCSWFFH |
| Consensus | ...Gyyp...qqsgq.qqgydspYRVsaE.QaAslkVAKaqqlaaQLPamCrlCGgDALsasQ........................... |

| | |
|---|---|
| | 1010 1020 1030 1040 |
| | ---------+---------+---------+---------\| |
| Ax1 | |
| Ax2 | |
| Dx5 | |
| HMW2(X03346) | |
| Dx7 | |
| Dy12 | |
| Dy10 | |
| Dy9 | |
| glu10(X03042) | LETPIMMQNEKLLQLKKEQIGCCIVCVACLISLCCILMIH |
| Consensus | ........................................ |

| | |
|---|---|
| | 1041 1050 1060 1065 |
| | --------+---------+----+ |
| Ax1 | |
| Ax2 | |
| Dx5 | |
| HMW2(X03346) | |
| Dx7 | |
| Dy12 | |
| Dy10 | |
| Dy9 | |
| glu10(X03042) | KYRGSIVVLSISKSVECNKEQKEMI |
| Consensus | ......................... |

TABLE 2

| | |
|---|---|
| 1 | atggcagagg atctgatcct ggagagatgt gatttgcagc tggaggtcaa |
| 51 | ggccgcgacc accgcacqgc cgacctgtgc cgggagaggc tggtgttgcg |
| 101 | gcggggccag cccttctggc tgacgctgca ctttgagggc cgtggctacg |
| 151 | aggctggtgt ggacactctc accttcaacg ctgtgaccgg cccagatccc |
| 201 | agtgaggagg ccgggactat ggcccggttc tcactgtcca gtgctgtcga |
| 251 | ggggggcacc tggtcagcct cagcagtgga ccagcaggac agcactgtct |
| 301 | cgctgctgct cagcacccca gctgatgccc ccattggcct gtatcgcctc |
| 351 | agcctggagg cctccactgg ttaccagggc tccagcttcg tactgggcca |
| 401 | cttcatcctg ctctacaatc ctcggtgccc agcggatgct gtctatatgg |
| 451 | actcagacca agagcggcag gagtatgtgc tcacccaaca gggcttcatc |
| 501 | taccagggct cggccaagtt catcaatggc ataccttgga acttcgggca |
| 551 | gtttgaagat gggatcctgg atatttgcct gatgctcttg gacaccaacc |
| 601 | ccaagttcct gaagaatgct ggccaagact gctcgcgccg cagcagacct |
| 651 | gtctacgtgg gccgggtggt gagcgccatg gtcaactgca atgacgatca |
| 701 | gggcgtgctt cagggacgct gggacaacaa ctacagtgat ggtgtcagcc |
| 751 | ccatgtcctg gatcggcagc gtggacatcc tgcggcgctg gaaagactat |
| 801 | gggtgccagc gcgtcaagta cggccagtgc tgggtcttcg ctgctgtggc |
| 851 | ctgcacagtg ctgcggtgcc ttggcatccc cacccgagtc gtgaccaact |
| 901 | ttaactcagc ccacgaccag aacagcaacc tgctcatcga gtacttccga |
| 951 | aacgagtctg gggagatcga ggggaacaag agcgagatga tctggaactt |
| 1001 | ccactgctgg gtggagtcgt ggatgaccag gccggacctg gagcctgggt |
| 1051 | acgaggggtg gcaggccctg gaccccacac cccaggagaa gagtgaaggg |
| 1101 | acatactgct gtggcccagt tccggttcga gccatcaagg agggccacct |
| 1151 | gaacgtcaag tatgatgcac ctttcgtgtt tgctgaggtc aatgctgacg |
| 1201 | tggtgaactg gatccggcag aaagatgggt ccctgcgcaa gtccatcaac |
| 1251 | catttggttg tggggctgaa gatcagtact aagagtgtgg gccgcgatga |
| 1301 | gcgagaggac atcacccaca cctacaagta cccagaggga tctgaagagg |
| 1351 | agcgggaagc ttttgttagg gccaaccacc taaataaact ggccacaaag |
| 1401 | gaagaggctc aggaggaaac gggagtggcc atgcggatcc gtgtgggcca |
| 1451 | gaacatgact atgggcagtg actttgacat ctttgcctac atcaccaatg |
| 1501 | gcactgctga gagccacgaa tgccaactcc tgctctgtgc acgcatcgtc |
| 1551 | agctacaatg gagtcctggg gcccgtgtgc agcaccaacg acctgctcaa |
| 1601 | cctgaccctg gatcccttct cggagaacag catccccctg cacatcctct |
| 1651 | atgagaagta cggtgactac ctgactgagt ccaacctcat caaggtgcga |
| 1701 | ggcctcctta tcgagccagc agccaacagc tatgtattgg ccgagaggga |
| 1751 | catttacctg gagaatccag aaatcaagat ccgggtcttg ggggagccca |
| 1801 | agcagaaccg caagctgatt gctgaggtgt ctctgaagaa tccgctccct |
| 1851 | gtgccgctgc tgggttgtat cttcaccgtg gaaggagctg gcctgaccaa |
| 1901 | ggaccagaag tcggtggagg tcccagaccc cgtggaagca ggggagcaag |
| 1951 | cgaaggtacg ggtggacctg ctgccgacgg aggtgggcct ccacaagctg |
| 2001 | gtggtgaact tcgagtgcga caagctgaag gccgtgaagg gctatcggaa |
| 2051 | cgtcatcatc ggccccgcct aa |

Table 2 shows the nucleotide sequence of the gene that codes for the guinea pig transglutaminase enzyme that we cloned starting from mRNA of liver and then sequenced. The underlined bases indicate the start and stop codons.

TABLE 3

| | |
|---|---|
| | Acd |
| | ˜˜˜˜˜˜ |
| | Apo1 |
| | ˜˜˜˜˜˜ |
| | Asp700 |
| | ˜˜˜˜˜˜˜˜˜˜˜ |
| | Xmnl |
| | ˜˜˜˜˜˜˜˜˜˜˜ |
| | EcoRi |
| | ˜˜˜˜˜˜ |
| 1 | GAATTCCTTC TACATCGGCT TAGGTGTAGC AACACGACTT TATTATTATT |
| | CTTAAGGAAG ATGTAGCCGA ATCCACATCG TTGTGCTGAA ATAATAATAA |
| | Bsmfl |
| | ˜˜˜˜˜ |
| 51 | ATTATTATTA TTATTATTAT TTTACAAAAA TATAAAATAG ATCAGTCCCT |
| | TAATAATAAT AATAATAATA AAATGTTTTT ATATTTTATC TAGTCAGGGA |
| 101 | CACCACAAGT AGAGCAAGTT GGTGAGTTAT TGTAAAGTTC TACAAAGCTA |
| | GTGGTGTTCA TCTCGTTCAA CCACTCAATA ACATTTCAAG ATGTTTCGAT |
| | Oral |
| | ˜˜˜˜˜˜ |
| 151 | ATTTAAAAGT TATTGCATTA ACTTATTTCA TATTACAAAC AAGAGTGTCA |
| | TAAATTTTCA ATAACGTAAT TGAATAAAGT ATAATGTTTG TTCTCACAGT |
| | Ndel |
| | ˜˜˜˜˜˜˜ |
| 201 | ATGGAACAAT GAAAACCATA TGACATACTA TAATTTTGTT TTTATTATTG |
| | TACCTTGTTA CTTTTGGTAT ACTGTATGAT ATTAAAACAA AAATAATAAC |
| | A [symbol] |
| | ˜˜˜ |
| 251 | AAATTATATA ATTCAAAGAG AATAAATCCA CATAGCCGTA AAGTTCTACA |
| | TTTAATATAT TAAGTTTCTC TTATTTAGGT GTATCGGCAT TTCAAGATGT |
| | A [symbol] HindIII |
| | ˜˜˜ ˜˜˜˜˜˜ |
| 301 | TGTGGTGCAT TACCAAAATA TATATAGCTT ACAAAACATG ACAAGCTTAG |
| | ACACCACGTA ATGGTTTTAT ATATATCGAA TGTTTTGTAC TGTTCGAATC |
| 351 | TTTGAAAAAT TGCAATCCTT ATCACATTGA CACATAAAGT GAGTGATGAG |
| | AAACTTTTTA ACGTTAGGAA TAGTGTAACT GTGTATTTCA CTCACTACTC |
| 401 | TCATAATATT ATTTTCTTTG CTACCCATCA TGTATATATG ATAGCCACAA |
| | AGTATTATAA TAAAAGAAAC GATGGGTAGT ACATATATAC TATCGGTGTT |
| | [symbol] 44l |
| | ˜˜˜˜˜˜˜ |
| | AspHI |
| | ˜˜˜˜˜˜˜ |
| | Bmyl |
| | ˜˜˜˜˜˜˜ |
| | BsiHKAl |
| | ˜˜˜˜˜˜˜ |
| | Bsp1286l |
| | ˜˜˜˜˜˜˜ |
| | HgiAl |
| | ˜˜˜˜˜˜˜ |
| | Snol |
| | ˜˜˜˜˜˜˜ |
| | Maelll EcoRV ApaU |
| | ˜˜˜˜˜ ˜˜˜˜˜˜˜ ˜˜˜˜˜˜˜ |
| 451 | AGTTACTTTG ATGATGATAT CAAAGAACAT TTTTAGGTGC ACCTAACAGA |
| | TCAATGAAAC TACTACTATA GTTTCTTGTA AAAATCCACG TGGATTGTCT |
| 501 | ATATCCAAAT AATATGACTC ACTTAGATCA TAATAGAGCA TCAAGTAAAA |
| | TATAGGTTTA TTATACTGAG TGAATCTAGT ATTATCTCGT AGTTCATTTT |
| 551 | CTAACACTCT AAAGCAACCG ATGGGAAAGC ATCTATAAAT AGACAAGCAC |
| | GATTGTGAGA TTTCGTTGGC TACCCTTTCG TAGATATTTA TCTGTTCGTG |
| | Foid |
| | ˜˜˜˜˜˜ |
| 601 | AATGAAAATC CTCATCATCC TTCACCACAA TTCAAATATT ATAGTTGAAG |
| | TTACTTTTAG GAGTAGTAGG AAGTGGTGTT AAGTTTATAA TATCAACTTC |
| | Tfil Mbo [symbol] |
| | ˜˜˜˜˜ ˜˜˜˜˜ |
| 651 | CATAGTAGTA GAATCCAACA ACAATGAAGA TCATTTTCGT ATTTGCTCTC |
| | GTATCATCAT CTTAGGTTGT TGTTACTTCT AGTAAAAGCA TAAACGAGAG |
| | BsrDI Hgal |
| | ˜˜˜˜˜˜˜ ˜˜˜˜˜˜ |
| | Mael |
| | ˜˜˜˜ |
| | Sphi Blal |
| | ˜˜˜˜˜˜ ˜˜˜˜ |
| 701 | CTTGCTATTG TTGCATGCAA TGCCTCTGCG TCTAGA |
| | GAACGATAAC AACGTACGTT ACGGAGACGC AGATCT |

TABLE 4

| Gene | Access | Sense primer | Cloning sites | Amplif. |
|---|---|---|---|---|
| Name | Number | Anti-sense primer | (5′-3′) | Dim. |

| | | | | |
|---|---|---|---|---|
| 1Ax1 | X61009 | PLT217-GCTCAGCAGAGTTCTATCACTGGCTGGCCAAC | BamHI-PstI | 2.783 |
| | | PLT219-GGATCCGATTACGTGGCTTTAGCAGACCGTC | | |
| 1Ax2 | M22208 | PLT228-GGATCCGCTTAGAAGCATTGAGTGGCCGC | BamHI-CelII | 2.910 |
| | | PLT230-GCTCAGCCTATCACTGGCTGGCCAACAATGC | | |
| 1Bx7 | M22209 | PLT185-TCTAGAATGGCACTACTCGACATGGTTAG | XabI-PstI | 2.853 |
| | | PLT186-CACCATGCAAGCTGCAGAGAG | | |
| 1Bx17 | JC2099 | PLT562-TCTAGATATGGCTAAGCGGTTAGTCCTC | XabI-SacI | 2.259 |
| | | PLT563-GATATCTGCGAGCTGCAGAGAGTTC | | |
| 1By9 | X61026 | PLT272-CCCGGGCACAGATAAATGTTGTGATTCA | XabI-SalI | 2.771 |
| | | PLT273-GTCGACTGCAAGTTGCAGAGAGTTCTAT | | |
| 1Dx5 | X12928 | G1B5-TGTTCCATGCAGGCTACCTCCCACTAC | EcoRI-SalI | 3.033 |
| | | PLT189-GTCGACATGCCTAAGCACCATGCGAG | | |
| 1Dy10 | X12929 | G2B3-AAGCTTTTCATTTTGCATTATTATTGGGTT | EcoRI-EcoRI | 2.555 |
| | | G2B5-ACCTTATCCATGCAAGCTACCTTCCAC | | |
| 1Dy12 | X03041 | PLT482-GAATTCGCAGATTTGCAAAAGCAATGGCTAAC | EcoRI-PstI | 3.035 |
| | | PLT483-TCTAGAGCTTGTGAGAAAGGGGTAATCATCAGTG | | |
| HMW2 | X03346 | PLT488-GAATTCAGCTTTGAGTGGCCGTAGATTTGCA | EcoRI-BamHI | 3.179 |
| | | PLT489-GGATCCATATAGGATCTGTCGCATTCATGGCTG | | |
| Glu1A | X03042 | PLT571-TCTAGATGGCTAAGCGGTTGGTCCTC | BamHI-SalI | 2.895 |
| | | PLT572-GATATCGCTCCTTGTTGCATTCAACACTCTTAC | | |
| TG | M19646 | PLT237-TCTAGAATGGCAGAGGATCTGATCCTGGAG | XbaI-SacI | 2.072 |
| | | PLT238-GAGCTCTTAGGCGGGGCCGATGATGACG | | |

[image]

1) A food flour with specific technological and nutritional characteristics, such as the ability to rise and low allergenicity;

2) A food flour, as in claim 1, derived from the seed of a plant obtained by inserting in its genome one or more genes that code for wheat storage proteins;

3) A food flour derived from the seed of a plant obtained by inserting in its genome one gene that codes for the transglutaminase enzyme;

4) A food flour derived from the seed of a plant obtained by inserting in its genome different combinations of the genes indicated in claims 2 and 3;

5) A food flour derived from the seed of a genetically modified plant of rice by inserting in its genome the genes according to claim 4;

6) A food flour derived from the seed of a genetically modified plant of corn by inserting in its genome the genes according to claim 4;

7) A food flour derived from the seed of a genetically modified plant of soybean by inserting in its genome the genes according to claim 4;

8) A nucleotide sequence that codes for the aminoacid sequence LKVAKAQQLAAQLPAMCR present in the C-terminal part of a class of storage proteins of wheat;

9) A recombinant plasmid including: (i) a DNA regulatory sequence of a specific plant; (ii) a structural sequence for storage proteins of wheat;

10) A recombinant plasmid including: (i) a DNA regulatory sequence of a specific plant, for example rice; (ii) a structural sequence of a storage protein of wheat, containing the aminoacid sequence of claim 8;

11) A recombinant plasmid including: (i) a DNA regulatory sequence of a specific plant; (ii) a structural sequence of wheat storage protein, genetically modified by means of techniques of protein engineering to eliminate specific epitopes;

12) A recombinant plasmid, according to claims 9 and 11, where the regulation sequence of the expression cassette comes from the gene corresponding to the wheat storage protein;

13) A recombinant plasmid, according to claims 9, where the regulation sequence of the expression cassette comes from the gene corresponding to the storage protein of a cereal or of a legume;

14) A recombinant plasmid, according to claim 9, where the structural sequence corresponds to the enzymatic protein transglutaminase.

15) Plant cells transformed with the DNA of one of the claims from 9 to 14;

16) Plant cells transformed with the DNA of two or more of the claims from 9 to 14 in different combinations;

17) Transgenic plants expressing the genes present in the plasmids of the claims from 9 to 14;

18) Transgenic plants expressing the genes present in two or more of the plasmids of the claims from 9 to 14;

19) The production of flours from the seeds of the plants of claims from 2 to 7, obtained with specific milling techniques;

20) The production of baked products obtained from flours or protein concentrates of claim 19;

21) The production of the transglutaminase enzyme from flours derived from the seeds of plants of claims 3, 4, 5, 6 and 7.
